(19) United States
(12) Patent Application Publication
(10) Pub. No.: US 2015/0336073 A1
(43) Pub. Date: Nov. 26, 2015

(54) METHODS AND APPARATUS FOR LIPID MULTILAYER PATTERNING

(71) Applicant: FLORIDA STATE UNIVERSITY RESEARCH FOUNDATION, INC., TALLAHASSEE, FL (US)

(72) Inventors: STEVEN LENHERT, TALLAHASSEE, FL (US); OMKAR A. NAFDAY, STAFFORD, TX (US); TROY W. LOWRY, TALLAHASSEE, FL (US)

(21) Appl. No.: 14/818,501

(22) Filed: Aug. 5, 2015

Related U.S. Application Data
(62) Division of application No. 13/417,588, filed on Mar. 12, 2012.
(60) Provisional application No. 61/451,619, filed on Mar. 11, 2011.
(60) Provisional application No. 61/451,635, filed on Mar. 11, 2011.

(51) Int. Cl. B01J 19/00; G01N 33/50; G01N 21/47

(52) U.S. Cl. CPC B01J 19/0046; G01N 21/4788; G01N 33/5008; B01J 2219/00621; B01J 2219/00734; B01J 2219/00382

(57) ABSTRACT

Described are methods and devices for forming patterned lipid multilayer structures on a substrate using a topographically structured stamp and a topographically structured brush.

### CROSS-REFERENCE TO RELATED APPLICATIONS

This application claims benefit of priority to the following applications: U.S. Provisional Application No. 61/451,635, to Lenhert et al., entitled “METHODS AND APPARATUS FOR LIPID MULTILAYER PATTERNING,” filed Mar. 11, 2011; U.S. Provisional Application No. 61/451,619, to Lenhert, entitled “IRIDESCENT SURFACES AND APPARATUS FOR REAL TIME MEASUREMENT OF LIQUID AND CELLULAR ADHESION,” filed Mar. 11, 2011, and to U.S. patent application Ser. No. ______ to Lenhert, entitled “IRIDESCENT SURFACES AND APPARATUS FOR REAL TIME MEASUREMENT OF LIQUID AND CELLULAR ADHESION,” filed Mar. 12, 2012, and the entire content and disclosures of these applications are incorporated herein by reference in their entirety.

### BACKGROUND

1. Field of the Invention

The present invention relates to lipid multilayer patterning.

2. Related Art

It has been difficult to form nanostructured lipid multilayers in particular patterns.

### SUMMARY

According to a first broad aspect, the present invention provides a method comprising the following steps: (a) printing one or more lipid inks on a substrate using a topographically structured stamp, and (b) removing the stamp from the substrate to form a patterned substrate, wherein the stamp comprises one or more recesses containing the one or more lipid inks prior to step (a), wherein the one or more recesses have one or more recess patterns, wherein the patterned substrate comprises one or more patterned arrays of lipid multilayer structures, and wherein the patterned arrays are based on the one or more recess patterns.

According to a second broad aspect, the present invention provides a device comprising: an ink palette on which is positioned one or more lipid inks, a stamp having one or more recesses for receiving the one or more lipid inks from the ink palette and printing the one or more lipid inks as patterned lipid multilayer structures on a substrate, an ink palette contacting device for causing the stamp to contact the ink palette, and a substrate contacting device for causing the stamp to contact the substrate.

According to a third broad aspect, the present invention provides a method comprising the following step: (a) spreading one or more lipid inks on a substrate using an edge of topographically structured brush to thereby form a patterned substrate comprising a patterned array of one or more lipid multilayer structures on the substrate, wherein the brush comprises one or more recesses in a surface of the brush including the edge, wherein the one or more recesses extend to the edge, and wherein the one or more recesses have a recess pattern that shape the one or more lipid inks to form the patterned array of one or more lipid multilayer structures.

According to a fourth broad aspect, the present invention provides a device comprising: a brush for spreading one or more lipid inks on a substrate to form a patterned substrate comprising a patterned array of one or more lipid multilayer structures on the substrate, wherein the brush comprises an edge and one or more recesses in a surface of the brush including the edge, wherein the one or more recesses extend to the edge, wherein the one or more recesses have a recess pattern that shape the one or more lipid inks to form the patterned array of one or more lipid multilayer structures, and wherein the brush is oriented at an angle of less than 90° with respect to a portion of the substrate on which the one or more lipid inks are present.

### BRIEF DESCRIPTION OF THE DRAWINGS

The accompanying drawings, which are incorporated herein and constitute part of this specification, illustrate exemplary embodiments of the invention and, together with the general description given above and the detailed description given below, serve to explain the features of the invention.

FIG. 1 is a schematic drawing of a method for printing lipid multilayers on a substrate according to one embodiment of the present invention.

FIG. 2 is a schematic drawing of an apparatus generating and analyzing patterned substrates according to one embodiment of the present invention.

FIG. 3 is a schematic drawing of an apparatus generating and analyzing patterned substrates according to one embodiment of the present invention.

FIG. 4 is an optical diffraction image of two dark circles printed (by hand) from a pipette tip onto a molded polydimethoxysilane (PDMS) diffraction grating according to one embodiment of the present invention.

FIG. 5 is an image of optical diffraction from flat glass surface that was patterned by printing two different lipid inks from a PDMS grating onto the flat glass surface.

FIG. 6 is an atomic force microscopy topographical image of lipid gratings printed using a topographically structured stamp onto a polystyrene surface (Petri dish) according to one embodiment of the present invention.

FIG. 7 is a schematic view showing a PDMS brush being made by cutting a PDMS grating stamp at a 45 degree angle at one end according to one embodiment of the present invention.

FIG. 8 is a schematic view showing the PDMS brush of FIG. 7 being used to spread an iridescent lipid ink on a surface of a substrate using a lower edge of the PDMS brush.

FIG. 9 is a schematic view showing the PDMS brush of FIG. 7 having spread iridescent lipid ink to form a lipid multilayer grating.

FIG. 10 is a schematic view showing the PDMS brush of FIG. 7 attached to a tip holder of a Dip-Pin Nanolithography® (DPN®) machine (Dip-Pen Nanolithography and DPN are registered trademarks of Nanoink).

FIG. 11 is an image, taken at a low angle, of light scattered from a flat surface that is patterned with lipid multilayers by dragging the lipid inked brush according to one embodiment of the present invention along the surface in the direction of the grating lines (i.e., brushing).

FIG. 12 is an image, taken at a high angle, of light scattered from a flat surface that is patterned with lipid multilayers by dragging the lipid inked brush according to one embodiment of the present invention along the surface in the direction of the grating lines (i.e., brushing).

FIG. 13 is a schematic perspective view of lipid spreading in a stamp according to one embodiment of the present invention.

FIG. 14 is a schematic top view of lipid spreading in a stamp according to one embodiment of the present invention.

FIG. 15 is an image of light diffracted from gratings with lipid spots spreading on the surface of a PDMS mold after 5 minutes of printing using DPN at high humidity.

FIG. 16 is an image of light diffracted from gratings with lipid spots spreading on the surface of a PDMS mold after 20 minutes of printing using DPN at high humidity.

FIG. 17 is an image of scattered light from a PDMS grating that was inked using a plastic pipette tip and shows lipid spread after 20 seconds.

FIG. 18 is an image of scattered light from the PDMS grating of FIG. 17 and shows lipid spread after 350 seconds.

FIG. 19 is an image of scattered light from a PDMS grating of FIG. 17 and shows lipid spread after 500 seconds.

FIG. 20 is an image of scattered light from a PDMS grating with lipids deposited by DPN before exposure to humidity.

FIG. 21 is an image of scattered light from the PDMS grating of FIG. 20 after exposure to high humidity.

FIG. 22 is a schematic illustration of a multilayer stamping process according to one embodiment of the present invention.

FIG. 23 is an image of a PDMS grating stamp according to one embodiment of the present invention.

FIG. 24 shows an atomic-force microscopy (AFM) height image of the spread of 1,2-dioleoyl-sn-glycero-3-phosphocholine (DOPC) multilayer film deposited on the grating stamp of FIG. 22 by DPN.

FIG. 25 shows DOPC spreading vertically along the recesses of the stamp of FIG. 22.

FIG. 26 shows a line trace of a region in FIG. 24 showing the 250 nm height of the PDMS stamp recesses together with the 65 nm height of DOPC multilayer deposited by DPN.

FIG. 27 shows the structure of DOPC.

FIG. 28 is an AFM height image of 22 μm wide DOPC diffraction grating stamped with the inked PDMS stamp on a polystyrene (PS) surface.

FIG. 29 is a close-up view of a region of FIG. 28 showing continuous DOPC lines spaced 555 nm apart that function as diffraction gratings.

FIG. 30 is a line trace of a region in FIG. 28 showing the similar height (38±9 nm) of the DOPC features created with different PDMS stamps.

FIG. 31 is a fluorescence microscopy image of two lipid inks patterned by DPN on a PDMS stamp according to one embodiment of the present invention.

FIG. 32 is a bright-field diffraction image of a first lipid stamped on a PS surface.

FIG. 33 is a bright-field diffraction image of a second lipid, different from the first lipid of FIG. 32, stamped on a PS surface.

FIG. 34 is a graph showing control over lipid multilayer height on different surfaces—polystyrene and freshly cleaned glass—with different PDMS stamps.

FIG. 35 is red diffraction obtained from stamped DPPC gratings with a 140 nm tall and 700 nm pitch stamp.

FIG. 36 is an optical micrograph with surface-enhanced ellipsometric contrast (SEEC) imaging of a white square region in FIG. 35 showing DPPC grating lines over a large area.

FIG. 37 is an AFM height image of a white square region 3602 in FIG. 36.

FIG. 38 is a line trace along a line in FIG. 37 showing an average height of 110±10 nm.

FIG. 39 is an image of the results of experiment where 16 different liposomal drug formulations arrayed onto a PDSM stamp and arrayed onto a glass surface.

FIG. 40 is a high magnification of an outlined part of FIG. 39 including spot 7 of FIG. 39.

### DETAILED DESCRIPTION OF THE PREFERRED EMBODIMENTS

### Definitions

Where the definition of a term departs from the commonly used meaning of the term, applicant intends to utilize the definitions provided below, unless specifically indicated.

For purposes of the present invention, it should be noted that the singular forms, “a,” “an” and “the” include reference to the plural unless the context as herein presented clearly indicates otherwise.

For purposes of the present invention, directional terms such as “top,” “bottom,” “upper,” “lower,” “above,” “below,” “left,” “right,” “horizontal,” “vertical,” “up,” “down,” etc., are used merely for convenience in describing the various embodiments of the present invention. The embodiments of the present invention may be oriented in various ways. For example, the diagrams, apparatuses, etc., shown in the drawing figures may be flipped over, rotated by 90° in any direction, reversed, etc.

For purposes of the present invention, a value or property is “based” on a particular value, property, the satisfaction of a condition, or other factor, if that value is derived by performing a mathematical calculation or logical decision using that value, property or other factor.

For purposes of the present invention, the term “analyte” refers to the conventional meaning of the term “analyte,” i.e., a substance or chemical constituent of a sample that is being detected or measured in a sample. In one embodiment of the present invention, a sample to be analyzed may be an aqueous sample, but other types of samples may also be analyzed using a device of the present invention.

For purposes of the present invention, the term “array” refers to a one-dimensional or two-dimensional set of microstructures. An array may be any shape. For example, an array may be a series of microstructures arranged in a line, such as an array of squares. An array may be arranged in a square or rectangular grid. There may be sections of the array that are separated from other sections of the array by spaces. An array may have other shapes. For example, an array may be a series of microstructures arranged in a series of concentric circles, in a series of concentric squares, a series of concentric triangles, a series of curves, etc. The spacing between sections of an array or between microstructures in any array may be regular or may be different between particular sections or between particular pairs of microstructures. The microstructure arrays of the present invention may be composed of microstructures having zero-dimensional, one-dimensional or two-dimensional shapes. The microstructures having two-dimensional shapes may have shapes such as squares, rectangles, circles, parallelograms, pentagons, hexagons, irregular shapes, etc.

For purposes of the present invention, the term “away” refers to increasing the distance between two aligned objects. For example, a contact controlling positioning device may be used to move: a stamp away from an ink palette, an ink palette away from a stamp, a stamp away from a substrate, a substrate away from a stamp, etc.

For purposes of the present invention, the term “biomolecule” refers to the conventional meaning of the term biomolecule, i.e., a molecule produced by or found in living cells, e.g., a protein, a carbohydrate, a lipid, a phospholipid, a nucleic acid, etc.

For purposes of the present invention, the term “brush” refers to a stamp-like object that is used to create lipid multilayers on the surface by being moved while in contact with the surface.

For purposes of the present invention, the term “camera” refers to any type of camera or other device that senses light intensity. Examples of cameras include digital cameras, scanners, charged-coupled devices, CMOS sensors, photomultiplier tubes, analog cameras such as film cameras, etc. A camera may include additional lenses and filters such as the lenses of a microscope apparatus that may be adjusted when the camera is calibrated.

For purposes of the present invention, the term “contacting surface” refers to a surface of a stamp or brush that contacts a surface onto which a pattern comprising lipid ink is to be printed.

For purposes of the present invention, the term “controlled environment chamber” refers to a chamber in which temperature and/or pressure and/or humidity can be controlled.

For purposes of the present invention, the term “dehydrated lipid multilayer grating” refers to a lipid multilayer grating that is sufficiently low in water content that it is no longer in fluid phase.

For purposes of the present invention, the term “detector” refers to any type of device that detects or measures light. A camera is a type of detector.

For purposes of the present invention, the term “dot” refers to a microstructure that has a zero-dimensional shape.

For purposes of the present invention, the term “fluorescence” refers to the conventional meaning of the term fluorescence, i.e., the emission of light by a substance that has absorbed light or other electromagnetic radiation of a different wavelength.

For purposes of the present invention, the term “fluorescent” refers to any material or mixture of materials that exhibits fluorescence.

For purposes of the present invention, the term “fluorescent dye” refers to any substance or additive that is fluorescent or imparts fluorescence to another material. A fluorescent dye may be organic, inorganic, etc.

For purposes of the present invention, the term “fluorescent microstructure” refers to a microstructure that is fluorescent. A fluorescent microstructure may be made of a naturally fluorescent material or may be made of a nonfluorescent material, such as a phospholipid, doped with a fluorescent dye.

For purposes of the present invention, the term “fluorescent nanostructure” refers to a nanostructure that is fluorescent. A fluorescent nanostructure may be made of a naturally fluorescent material or may be made of a nonfluorescent material, such as a phospholipid, doped with a fluorescent dye.

For purposes of the present invention, the term “fluid” refers to a liquid or a gas.

For purposes of the present invention, the term “freezing by dehydration” refers to removal of residual water content, for instance by incubation in an atmosphere with low water content, for instance a vacuum (<50 mbar) or at relative humidity below 40% (at standard temperature and pressure).

For purposes of the present invention, the term “grating” refers to an array of dots, lines, or a 2D shape that are regularly spaced at a distance that causes coherent scattering of incident light.

For purposes of the present invention, the term “groove” refers to an elongated recess in a stamp or brush. A groove is not limited to a linear groove, unless clearly specified otherwise in the description below. The dimensions of a groove may change depending on the depth of the groove. For example, a groove may be wider at the top of the groove than at the bottom of the groove, such as in a V-shaped groove.

For purposes of the present invention, the term “groove pattern” refers to the pattern made by one or more grooves of a stamp or brush.

For purposes of the present invention, the term “height” refers to the maximum thickness of the microstructure on a substrate, i.e., the maximum distance the microstructure projects above the substrate on which it is located.

For purposes of the present invention, the term “high humidity atmosphere” refers to an atmosphere having a relative humidity of 40% or greater.

For purposes of the present invention, the term “iridescent” refers to any structure that scatters light.

For purposes of the present invention, the term “iridescent microstructure” refers to a microstructure that is iridescent.

For purposes of the present invention, the term “iridescent nanostructure” refers to a nanostructure that is iridescent.

For purposes of the present invention, the term “irregular pattern” refers to a pattern of ridges and recesses that are not organized in a specific geometric pattern. For example, ridges and or recesses printed to resemble a picture of a human face, a picture of a leaf, a picture of an ocean wave, etc. are examples of irregular patterns. Using photolithography, almost any type of pattern for recesses and/or ridges may be formed in a stamp or brush of the present invention.

For purposes of the present invention, the term “light,” unless specified otherwise, refers to any type of electromagnetic radiation. Although, in the embodiments described below, the light that is incident on the gratings or sensors is visible light, the light that is incident on the gratings or sensors of the present invention may be any type of electromagnetic radiation, including infrared light, ultraviolet light, etc., that may be scattered by a grating or sensor. Although, in the embodiments described below, the light that is scattered from the gratings or sensors and detected by a detector is visible light, the light that is scattered by a grating or sensor of the present invention and detected by a detector of the present invention may be any type of electromagnetic radiation, including infrared light, ultraviolet light, etc. that may be scattered by a grating or sensor.

For purposes of the present invention, the term “light source” refers to a source of incident light that is scattered by a grating or sensor of the present invention. In one embodiment of the present invention, a light source may be part of a device of the present invention. In one embodiment a light source may be light present in the environment of a sensor or grating of the present invention. For example, in one embodiment of the present invention a light source may be part of a device that is separate from the device that includes the sensors and detector of the present invention. A light source may even be the ambient light of a room in which a grating or sensor of the present invention is located. Examples of a light source include a laser, a light-emitting diode (LED), an incandescent light bulb, a compact fluorescent light bulb, a fluorescent light bulb, etc.

For purposes of the present invention, the term “line” refers to a “line” as this term is commonly used in the field of nanolithography to refer to a one-dimensional shape.

For purposes of the present invention, the term “lipid” refers to hydrophobic or amphipilic molecules, including but not limited to biologically derived lipids such as phospholipids, triacylglycerols, fatty acids, cholesterol, or synthetic lipids such as surfactants, organic solvents, oils, etc.

For purposes of the present invention, the term “lipid ink” refers to any material comprising a lipid applied to a stamp.

For purposes of the present invention, the term “lipid multilayer” refers to a lipid coating that is thicker than one molecule.

For purposes of the present invention, the term “lipid multilayer grating” refers to a grating comprising lipid multilayers.

For purposes of the present invention, the term “lipid multilayer structure” refers to a structure comprising one or more lipid multilayers. A lipid multilayer structure may include a dye such as a fluorescent dye.

For purposes of the present invention, the term “low humidity atmosphere” refers to an atmosphere having a relative humidity of less than 40%.

For purposes of the present invention, the term “lyotropic” refers to the conventional meaning of the term “lyotropic,” i.e., a material that forms liquid crystal phases because of the addition of a solvent.

For purposes of the present invention, the term “microfabrication” refers to the design and/or manufacture of microstructures.

For purposes of the present invention, the term “microstructure” refers to a structure having at least one dimension smaller than 1 mm. A nanostructure is one type of microstructure.

For purposes of the present invention, the term “nanofabrication” refers to the design and/or manufacture of nanostructures.

For purposes of the present invention, the term “neat lipid ink” refers to a lipid ink consisting of a single pure lipid ink.

For purposes of the present invention, the term “nanostructure” refers to a structure having at least one dimension on the nanoscale, i.e., a dimension between 0.1 and 100 nm.

For purposes of the present invention, the term “patterned substrate” refers to a substrate having a patterned array of lipid multilayer structures on at least one surface of the substrate.

For purposes of the present invention the term “palette” refers to a substrate having one or more lipid inks that are made available to be picked up or drawn into the recesses or other topographical or chemical features of a stamp. The one or more lipid inks may be located in recesses, inkwells, etc. in the palette, or deposited onto a flat palette.

For purposes of the present invention, the term “plurality” refers to two or more. So an array of microstructures having a “plurality of heights” is an array of microstructures having two or more heights. However, some of the microstructures in an array having a plurality of heights may have the same height.

For purposes of the present invention, the term “recess” refers to a recess of any size or shape in a stamp or brush. A recess may have any cross-sectional shape such as a line, a rectangle, a square, a circle, an oval, etc. The dimensions of a recess may change depending on the depth of the recess. For example, a recess may be wider at the top of the recess than at the bottom of the recess, such as in a V-shaped recess.

For purposes of the present invention, the term “recess pattern” refers to the pattern made by one or more recesses of a stamp or brush.

For purposes of the present invention, the term “regular pattern” refers to a pattern of ridges and recesses organized in a specific geometric pattern. For example, a series of parallel recesses and/or lines is one example of a regular pattern. One or more arrays of ridges and recesses arranged in a square, a circle, an oval, a star, etc. is another example of a regular pattern.

For purposes of the present invention, the term “patterned array” refers to an array arranged in a pattern. A patterned array may comprise a single patterned array of lipid multilayer structures or two or more patterned arrays of lipid multilayer structures. Examples of patterned arrays of lipid multilayer structures are a patterned array of dots, a patterned array of lines, a patterned array of squares, etc.

For purposes of the present invention, the term “ridge” refers to any raised structure. A ridge is not limited to a linear ridge, unless clearly specified otherwise in the description below. A ridge may have any cross-sectional shape such as a line, a rectangle, a square, a circle, an oval, etc. The dimensions of a ridge may change depending on the depth of a neighboring groove. For example, a ridge may be wider at the bottom of the ridge than at the top of the ridge, such as in a V-shaped ridge. A ridge may constitute the entire contacting surface of a stamp or brush after recesses have been formed, etched, etc. into the stamp or brush.

For purposes of the present invention, the term “scattering” and the term “light scattering” refer to the scattering of light by deflection of one or more light rays from a straight path due to the interaction of light with a grating or sensor. One type of interaction of light with a grating or sensor that results in scattering is diffraction.

For purposes of the present invention, the term “sensor” and the term “sensor element” are used interchangeably, unless specified otherwise, and refer to a material that may be used to sense the presence of an analyte.

For purposes of the present invention, the term “square” refers to a microstructure that is square in shape, i.e., has a two-dimensional shape wherein all sides are equal.

For purposes of the present invention, the term “topographically structured brush” refers to a brush having recesses that form one or more recess patterns.

For purposes of the present invention, the term “topographically structured stamp” refers to a stamp having recesses that form one or more recess patterns.

For purposes of the present invention, the term “toward” refers to decreasing the distance between two aligned objects. For example, a contact controlling positioning device may be used to move: a stamp towards an ink palette, an ink palette towards a stamp, a stamp towards a substrate, a substrate towards a stamp, etc.

### Description

A lipid multilayer is a structure comprising lipids that is more than one molecule thick. Liposomes, which are lipid-based nano- and microparticles and are widely used for drug delivery, fit this definition because liposomes are three-dimensional compartments enclosed by at least one lipid bilayer, such that the entire liposome is at least two bilayers thick. Methods for patterning lipid multilayers have only recently been developed. These include DPN,⁷ dewetting on a prepatterned surface,⁸ and photothermal patterning.⁹ Micro- and nanostructured lipid multilayers on surfaces hold the promise of combining certain properties of solution-based liposomes with surface-based capabilities. In particular, material can be encapsulated in surface-supported lipid multilayers, and lipid composition can be varied on the same surface in a microarray format for screening applications.⁷ Furthermore, entirely new properties are made possible by the controlled formation of lipid multilayer nanostructures. For example, control of the iridescent optical properties of lipid multilayer structures formed by DPN has been demonstrated.^{7a-7d} In one approach, controlling the thickness of a lipid multilayer film between 1 and 100 nanometers allowed tuning of the iridescent color of the film caused by thin-film interference.^{7b} In another application made possible by control of both the lateral and vertical dimensions of surface-supported lipid multilayers, fluid diffraction gratings composed of fluid lipids were fabricated.^{7a} In the case of diffraction gratings, the spacing of the lines in a grating determines which wavelengths are visible at which angles, whereas the thickness of the gratings determines the efficiency of optical diffraction. The challenge in the fabrication of lipid multilayer gratings that DPN was able to solve was to generate structures with small lateral pitch (on the order of the wavelength of visible light, e.g., <700 nm), yet with higher multilayer thicknesses of ˜50 nm.^{7a} When functional lipids were incorporated into the lipid multilayer gratings and they were immersed in water, a label-free biosensor was demonstrated where the diffraction efficiency changed in response to analyte binding.^{7a} These materials have the potential to permit massively multiplexed sensor arrays, provided that a scalable method can be developed for their fabrication out of multiple materials over large areas.

DPN is a versatile method for deposition of different nanomaterials in close proximity at specific sites¹⁰ on diverse surfaces.¹¹ Although DPN is ideally suited for the creation of prototype diffraction gratings^{7a} and can also be carried out in a massively parallel and multiplexed fashion,^{7b,12} its ability to integrate more than 3 materials in a uniform manner is still limited by fabrication time and uniformity between ink transport rates of different tips in parallel arrays. In general, because of theoretical and practical constraints of nonuniform ink coating (leading to nonuniform ink flow), ink depletion, writing time, and tuning surface chemistry in DPN, the processing rate cannot be increased much beyond the typical rate of 1 μm² min⁻¹ per tip, and the aspect ratio (height/width) of topographical features is limited.^{13,11a} Moreover, DPN is limited in the types of lipids that can be patterned—only phospholipids like 1,2-dioleoyl-sn-glycero-3-phosphocholine (DOPC) that have a low gel-liquid phase transition (−20° C.) can be used to coat the tip as they are fluid at room temperature. Although fluid-phase lipids are useful, immersion of such gratings in water for biological applications requires thoroughly dehydrating them, which poses technical problems. A method that could pattern gel phase lipid multilayers could solve this problem.

In contrast to DPN, approaches based on using polydimethoxysilane (PDMS), such as microcontact printing and polymer-pen lithography, provide faster, cheaper and easier ways to create patterns over large areas.¹⁴ For example, in polymer pen lithography, an array of polymer tips is created that can have a tip density (250 000 per cm²) higher than that of cantilever-based tips of DPN.¹⁵ PDMS stamps covering large areas can be cheaply fabricated in one step from a silicon master. Microcontact printing is a mature technology and has been used to create structures with diverse applications whose features are defined by the topography of the stamp, for example, lipid bilayer patterning,^{5a} protein patterning,¹⁶ biosensing,¹⁷ and screening drug-membrane interactions.^{5b} Multilayers have been created with microcontact printing with polyelectrolytes,¹⁸ nanofibers,¹⁹ and nanoparticles,²⁰ and multiplexed (i.e., multimaterial) microcontact printing has been demonstrated by inking of the PDMS stamp with more than one material and then printing.²¹ Importantly, multilayered alkoxysilane optical gratings have been fabricated by microcontact printing, and DPN has been used to ink flat stamps for fabrication of chemical patterns.²² Another approach to generating topographical structures is nanoimprint lithography, which involves an embossing process capable of making nanometer-scale topographical structures.²³ Here we describe a method that combines the lateral patterning capabilities and scalability of microcontact printing with the topographical control of nanoimprint lithography, and the multimaterial integration aspects of dip pen nanolithography in order to create nanostructured lipid multilayer arrays. We refer to this approach as multilayer stamping.

Microstructured and nanostructured lipid multilayers on surfaces are a promising biofunctional nanomaterial. For example, surface-supported lipid multilayer diffraction gratings with optical properties that depend on the microscale spacing of the grating lines and the nanometer thickness of the lipid multilayers have been fabricated previously by DPN, with immediate applications as label-free biosensors. The innate biocompatibility of such gratings makes them promising as biological sensor elements, model cellular systems, and construction materials for nanotechnology.

Lipid multilayer gratings are lipid multilayer microstructures with potential applications as multiplexed biosensing elements, see S. Lenhert, C. A. Mirkin, H. Fuchs, In situ lipid dip-pen nanolithography under water, Scanning 31, 1-9 (2010), the entire contents and disclosure of which are incorporated herein by reference. Parallel and multiplexed DPN may be used to deposit multiple lipids simultaneously with controllable multilayer heights, laterally structured to form arbitrary patterns (e.g., diffraction gratings) with feature sizes on the same scale as visible light. In situ observation of the light diffracted from the patterns can be carried out during DPN and used for optical quality control without the need for fluorescent labels. Although diffraction gratings are one of the simplest and best-studied photonic structures, lipid multilayer gratings are a fundamentally new type of material because they are fluid, innately biocompatible, and immersible in water.

The interaction of electromagnetic waves with matter can be controlled by structuring the matter on the scale of the wavelength of light, and various photonic components have been made by structuring materials using top-down or bottom-up approaches. Dip-pen nanolithography is a scanning-probe-based fabrication technique that may be used to deposit materials on surfaces with high resolution and, when carried out in parallel, with high throughput.

Fundamental photonic components can be generated from a large variety of materials by top-down lithography or bottom-up self-assembly. Examples include simple Bragg gratings, stacks and two- or three-dimensional photonic materials. A major challenge lies in the integration of multiple chemical functionalities for the generation of more complex devices, including the readout system, in a simple and efficient way. Top-down microfabrication strives to fabricate smaller structures from a single material, whereas the bottom-up approach seeks to assemble and integrate small components into larger and more complex devices. DPN is a unique method of microfabrication and nanofabrication, as it is a direct-write method that allows the bottom-up integration of a variety of materials (especially organic and biological molecules) with both high resolution and high throughput, see Ginger, D. S., Zhang, H. & Mirkin, C. A., The evolution of dip-pen nanolithography, Agnew. Chem. Int. Ed, 43, 30-45 (2004) and Salaita, K., Wang, Y. H. & Mirkin, C. A., Applications of dip-pen nanolithography, Nature Nanotech. 2, 145-155 (2007), the entire contents and disclosures of which are incorporated herein by reference.

Phospholipids are fundamental structural and functional components of biological membranes that are both fluid and responsive to external stimuli. Phospholipids in biological systems form the bilayer structure of cellular membranes, as well as a variety of multilayer structures. Examples of lipid multilayers in biological systems include multilamellar cristae in the mitochondria, thylakoid grana and the cisternae of the Golgi apparatus and endoplasmic reticulum. Synthetic phospholipid multilayers can be fabricated by spin-coating, see Mathieu M., Schunk D., Franzka S., Mayer C. and Hartmann N. 2010 J. Vac. Sci. Technol. A 28 953; Mennicke U. and Salditt T., 2002 Langmuir 18 8172; controlling hydration between glass slides, see Trapp M., Gutberlet T., Juranyi F., Unruh T., Deme B., Tehei M. and Peters J., 2010 J. Chem. Phys. 133 164505 Eggeling C. et al., 2009 Nature 457 1159; Langmuir-Blodgett deposition, see Pompeo G., Girasole M., Cricenti A., Cattaruzza F., Flamini A., Prosperi T., Generosi J. and Castellano A. C., 2005 Biomembranes 1712 29; laser writing, see Scheres L., Klingebiel B., ter Maat J., Giesbers M., de Jong H., Hartmann N. and Zuilhof H., 2010 Small 6 1918; dewetting, see Le Berre M., Chen Y. and Baigl D., 2009 Langmuir 25 2554; Diguet A., Le Berre M., Chen Y. and Baigl D., 2009 Small 5 1661; and DPN, see Lenhert S., Sun P., Wang Y. H., Fuchs H. and Mirkin C. A., 2007 Small 3 71, and the entire contents and disclosures of the above articles are incorporated herein by reference.

In the presence of water, phospholipids spontaneously self-organize to form liposomes (or vesicles), which are widely used for a variety of biological and nanotechnological applications. For example, the physical chemistry of liposome adhesion on surfaces is well-studied as a model system for cell-surface interactions and surface biofunctionalization in general. Furthermore, liposomes have been used as nanoscale containers with attoliter to zeptoliter volumes and networks for nanoscale transport of materials between vessels. The loading of vesicles (for example, by surface binding, encapsulation or intercalation) with a variety of biofunctional materials such as drugs, nucleic acids and proteins is developed for applications in delivery to biological cells.

DPN has emerged as a reliable method for creating microstructures with a wide variety of materials on desired surfaces, see Lenhert S. et al., 2010 Nat. Nanotechnol. 5 275; Braunschweig A. B., Huo F. W. and Mirkin C. A., 2009 Nat. Chem. 1 353; Lenhert S., Fuchs H. and Mirkin C. A., 2009 Materials Integration by Dip-pen Nanolithography (Weinheim: Wiley-VCH); Zhang H., Amro N., Disawal S., Elghanian R., Shile R., and Fragala J., 2007 Small 3 81; Li B., Goh C. F., Zhou X. Z., Lu G., Tantang H., Chen Y. H., Xue C., Boey F. Y. C. and Zhang H., 2008 Adv. Mater. 20 4873; Li H., He Q. Y., Wang X. H., Lu G., Liusman C., Li B., Boey F., Venkatraman S. S. and Zhang H., 2011 Small 7 226; Salaita K., Wang Y. H. and Mirkin C. A., 2007 Nat. Nanotechnol. 2 145; Haaheim J. and Nafday O. N., 2008 Scanning 30 137; and Ginger D. S., Zhang H. and Mirkin C. A., 2004 Angew. Chem. Int. Ed. 43 30, the entire contents and disclosures of which are incorporated herein by reference. Using phospholipids as the ink for DPN allows control of the lipid multilayer stacking (height) and biocompatible material integration on solid surfaces, see Sekula S. et al., 2008 Small 4 1785; and Wang Y. H., Giam L. R., Park M., Lenhert S., Fuchs H. and Mirkin C. A. 2008 Small 4 1666, the entire contents and disclosures of which are incorporated herein by reference.

The resulting biomimetic lipid structures may be used in cell-surface models, biochemical sensors, drug screening and delivery vehicles, for analysis of cell-cell interactions, and to elucidate the mechanisms of membrane trafficking. Lipid multilayer structures have been fabricated using both serial and massively parallel DPN modes, allowing throughputs on the scale of cm² min⁻¹. The height of phospholipid structures can be tuned by the tip contact time and controlling the relative humidity of the patterning environment in DPN, see Lenhert S., Sun P., Wang Y. H., Fuchs H. and Mirkin C. A. 2007 Small 3 71, the entire contents and disclosure of which are incorporated herein by reference.

In one embodiment, the present invention provides a method for rapid creation of lipid multilayer microstructures and nanostructures over large surface areas.

In one embodiment, the present invention provides a method that is cheap, fast, capable of multiplexing, customizable, versatile and capable of patterning a wider variety of lipids with higher throughput than traditional lipid DPN.

In one embodiment, the present invention provides a method that combines the unique advantages of DPN and μ-CP techniques to create biocompatible nanostructures with controlled dimensions.

In one embodiment, the present invention provides a sensor that employs the diffraction change upon the interaction of a prescription drug with the lipid multilayer.

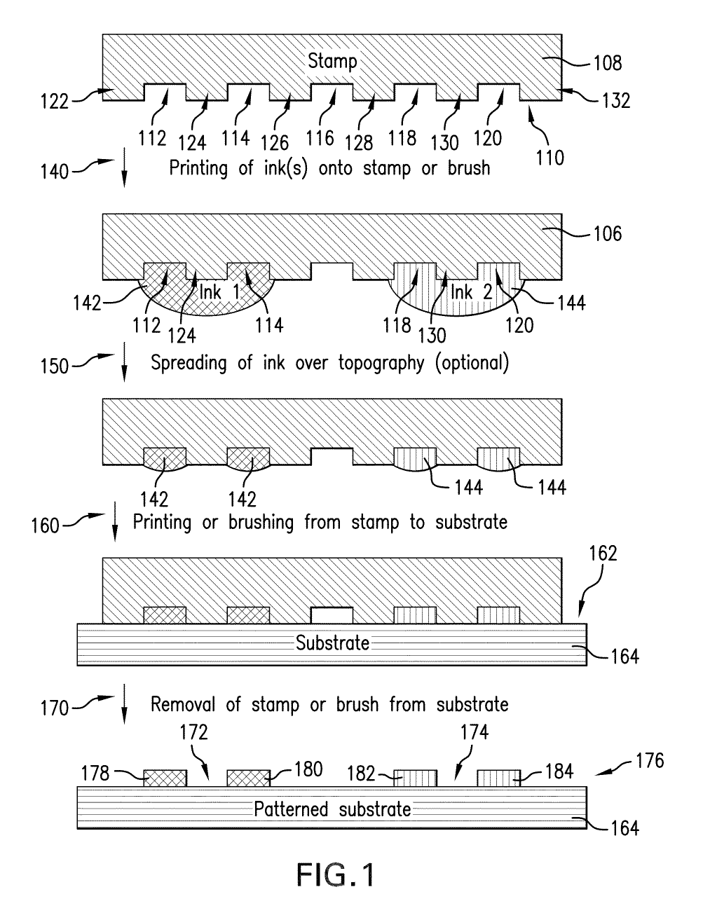

FIG. 1 shows a method for inking a topographically structured stamp, and printing lipid multilayers from the stamp onto a substrate to form a patterned substrate according to one embodiment of the present invention. As shown in FIG. 1, topographically structured stamp 108 has a topographically structured surface 110 that includes grooves 112, 114, 116, 118 and 120 and ridges 122, 124, 126, 128, 130 and 132. In step 140, neat lipid inks 142 and 144 are applied onto a topographically structured surface 110 of topographically structured stamp 108. In one embodiment of the present invention, step 140 may involve topographically structured stamp 108 contacting an ink palette (not shown) on which neat lipid ink 142 and neat lipid ink 142 are present. When ridges 122, 124, 126, 128, 130 and 132 of topographically structured stamp 108 contact the ink pallet, neat lipid ink 142 is forced into grooves 112 and 114 and neat lipid ink 144 is forced into grooves 118 and 120. When topographically structured stamp 108 is lifted up from the ink pallet, topographically structured stamp 108 picks up neat lipid ink 142 and 144. Neat lipid ink 142 partially fills grooves 112 and 114 and covers part of ridge 124. Neat lipid ink 144 partially fills grooves 118 and 120 and covers part of ridge 130. In step 150 lipid inks 142 and 144 are spread on topographically structured surface 110 of topographically structured stamp 108. Step 150 results in lipid ink 142 more completely filling grooves 112 and 114 and uncovering ridge 124 and lipid ink 144 more completely filling grooves 118 and 120 and uncovering ridge 130. Step 150 results in changing the diffraction properties of lipid ink 142 and lipid ink 144, which may be monitored in real time by diffraction imaging. In step 160, topographically structured surface 110 of topographically structured stamp 108 is brought into contact with a surface 162 of a substrate 164 to be patterned. In step 170, topographically structured stamp 108 is removed leaving patterned arrays 172 and 174 on substrate 164, thereby forming patterned substrate 176. Patterned array 172 comprises lipid multilayer structures 178 and 180 made from lipid ink 142. Patterned array 174 comprises lipid multilayer structures 182 and 184 made from lipid ink 144.

In one embodiment of the present invention the topographically structured stamp in may be made of molded PDMS diffraction gratings. As shown in FIG. 1, multiple lipids can be applied onto the same stamp. However, in some embodiments of the present invention, only one lipid may be applied to the same stamp.

In one embodiment of the present invention, the lipid ink used in the method shown in FIG. 1 may be a lipid ink and/or lipids mixed with other molecules.

The substrate may be any material on which lipid materials may be deposited including glass, plastic, etc. In one embodiment of the present invention, the substrate may be polystyrene (PS), such as a PS Petri dish.

Although one type of array of lipid multilayer structures, i.e., lines, are shown in FIG. 1, the lipid multilayer structures of the present invention may be any shape.

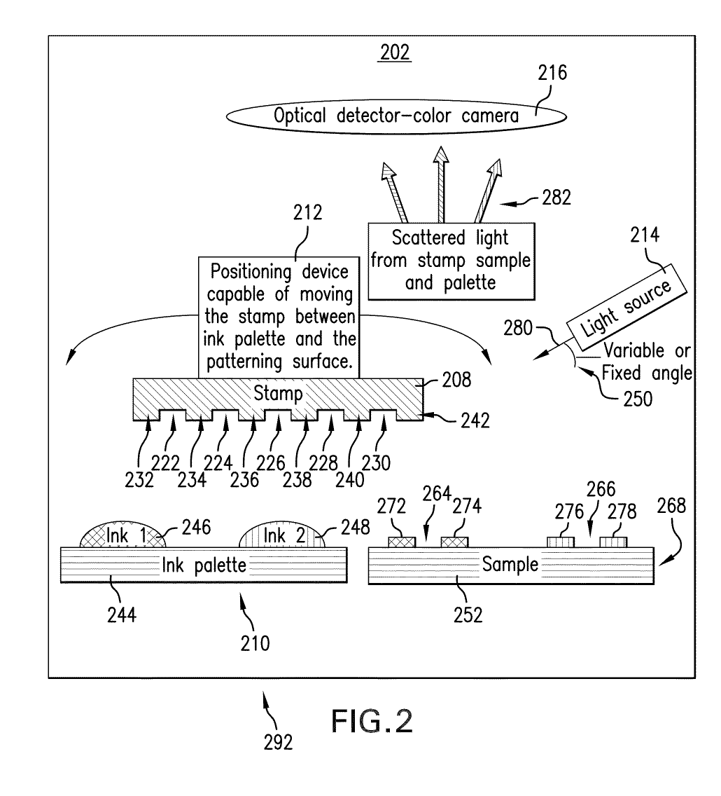

In one embodiment of the present invention, an apparatus may be used to pick up inks from a palette and deposit the inks onto a sample substrate for pattern generation. A motorized positioning device may be used to move the stamp between different positions. The process can be monitored in real time using a light source, and, in the case of iridescent structure formation, scattered light from the surface may be quantified using an optical detection system. An example of such an apparatus is shown in FIG. 2. FIG. 2 shows apparatus 202 for forming and analyzing patterned substrates according to one embodiment of the present invention. Apparatus 202 comprises a topographically structured stamp 208, an ink palette 210, a stamp positioning device 212, a light source 214 and an optical detector 216. Topographically structured stamp 208 includes grooves 222, 224, 226, 228 and 230 and ridges 232, 234, 236, 238, 240 and 242. Ink palette 210 includes a palette substrate 244 on which is deposited two different lipid inks, i.e., lipid inks 246 and 248. Light source 214 is positioned at an angle 250 that may be adjusted. Stamp positioning device 212 is used to move topographically structured stamp 208 both horizontally and vertically. In order to pick up lipid inks 246 and 248 from ink palette 210, stamp positioning device 212 positions topographically structured stamp 208 above ink palette 210. Topographically structured stamp 208 is then lowered by stamp positioning device 212 (moved towards stamp positioning device 212) so that ridges 232, 234, 236, 238, 240 and 242 of topographically structured stamp 208 contact ink palette substrate 244. When ridges 232, 234, 236, 238, 240 and 242 of topographically structured stamp 108 contact ink pallet substrate 244, lipid ink 246 is forced into grooves 222 and 224 and lipid ink 248 is forced into grooves 228 and 230. When topographically structured stamp 226 is lifted up from the ink pallet 210 by stamp positioning device 212, topographically structured stamp 208 picks up lipid ink 246 and lipid ink 248. Stamp positioning device 212 then positions topographically structured stamp 208 above a sample substrate 252. Stamp positioning device 212 then lowers topographically structured stamp 208 (moves topographically structured stamp 208 towards sample substrate 252) to contact sample substrate 252. Stamp positioning device 212 then raises topographically structured stamp 208 (moves topographically structured stamp 208 away from sample substrate 252) to thereby deposit patterned array 264 made of lipid ink 246 from grooves 222 and 224 and patterned array 266 made of lipid ink 248 from grooves 228 and 230 to form a patterned substrate 268. Patterned array 264 is a diffraction grating comprising lipid multilayer lines 272 and 274. Patterned array 266 is a diffraction grating comprising lipid multilayer lines 276 and 278. Light source 214 may positioned to shine light 280 on patterned substrate 268 that is scattered by patterned arrays 264 and 266 as scattered light 282 and detected by optical detector 216. Light source 214 may also be positioned to shine light 280 on ink palette 210 that is scattered by patterned lipid ink 246 and 248 on ink palette 210 and detected by optical detector 216. Apparatus 202 is contained in a controlled environment chamber 292 in which temperature, pressure and humidity are controlled.

The stamp positioning device may be a motorized positioning stage, similar to a mask aligner in photolithography, which is capable of moving the stamp in three dimensions (as well as controlling the relative tilt angles) relative to the substrate by motors, and also equipped with an optical monitoring system such as a camera.

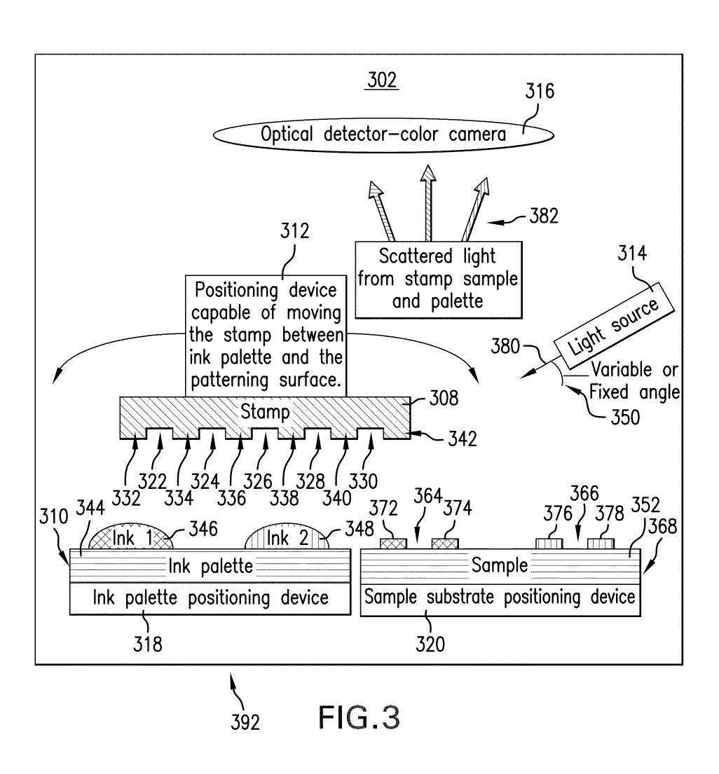

Although in the apparatus of FIG. 3, the stamp is moved up and down relative to ink palette and sample substrate, in other embodiments of the present invention the ink palette and sample substrate may be moved up and down relative to the stamp. FIG. 3 shows an example of such an apparatus. FIG. 3 shows apparatus 302 for forming and analyzing patterned substrates according to one embodiment of the present invention. Apparatus comprises a topographically structured stamp 308, an ink palette 310, a stamp positioning device 312, a light source 314, an optical detector 316, an ink palette contact controlling positioning device 318 and a sample substrate contact controlling positioning device 320. Topographically structured stamp 308 includes grooves 322, 324, 326, 328 and 330 and ridges 332, 334, 336, 338, 340 and 342. Ink palette 310 includes a palette substrate 344 on which are deposited two different lipid inks, i.e., lipid inks 346 and 348. Light source 314 is positioned at an angle 350 that may be adjusted. Positioning device 312 is used to move topographically structured stamp 308 both horizontally and vertically. In order to pick up lipid inks 346 and 348 from ink palette 310, stamp positioning device 312 positions topographically structured stamp 308 above ink palette 310. Ink palette 310 is then raised by ink palette positioning device 318 (moved towards topographically structured stamp 308) so that ridges 332, 334, 336, 338, 340 and 342 of topographically structured stamp 308 contact ink palette substrate 344. When ridges 332, 334, 336, 338, 340 and 342 of topographically structured stamp 108 contact ink pallet substrate 344, lipid ink 346 is forced into grooves 322 and 324 and lipid ink 348 is forced into grooves 328 and 330. When ink palette 310 is lowered by ink palette contact controlling positioning device 318 (moves ink palette 310 away from topographically structured stamp 308), topographically structured stamp 308 picks up lipid inks 346 and 348. Stamp positioning device 312 then positions topographically structured stamp 308 above a sample substrate 352. Sample substrate positioning device 320 raises sample substrate 352 (moves sample substrate 352 towards topographically structured stamp 308) until sample substrate 352 contacts topographically structured stamp 308. Sample substrate contact controlling positioning device 320 then lowers sample substrate 352 (moves sample substrate away from topographically structured stamp 308) so that topographically structured stamp 208 deposits patterned array 364 made of lipid ink 346 from grooves 322 and 324 and patterned array 366 made of lipid ink 348 from grooves 328 and 330 to form a patterned substrate 368. Patterned array 364 is a diffraction grating comprising lipid multilayer lines 372 and 374. Patterned array 366 is a diffraction grating comprising lipid multilayer lines 376 and 378. Light source 314 may be positioned to shine light 380 on patterned substrate 368 that is scattered by patterned arrays 364 and 366 as scattered light 382 and detected by optical detector 316. Light source 314 may also be positioned to shine light 380 on ink palette 310 that is scattered by patterned lipid ink 346 and 348 on ink palette 310 and detected by optical detector 316. Apparatus 302 is contained in a controlled environment chamber 392 in which temperature, pressure and humidity are controlled.

Although the apparatuses of FIGS. 2 and 3 are shown in a particular orientation for simplicity of illustration, the apparatuses may be oriented in any direction including upside down, at an angle, rotated 90°, etc.

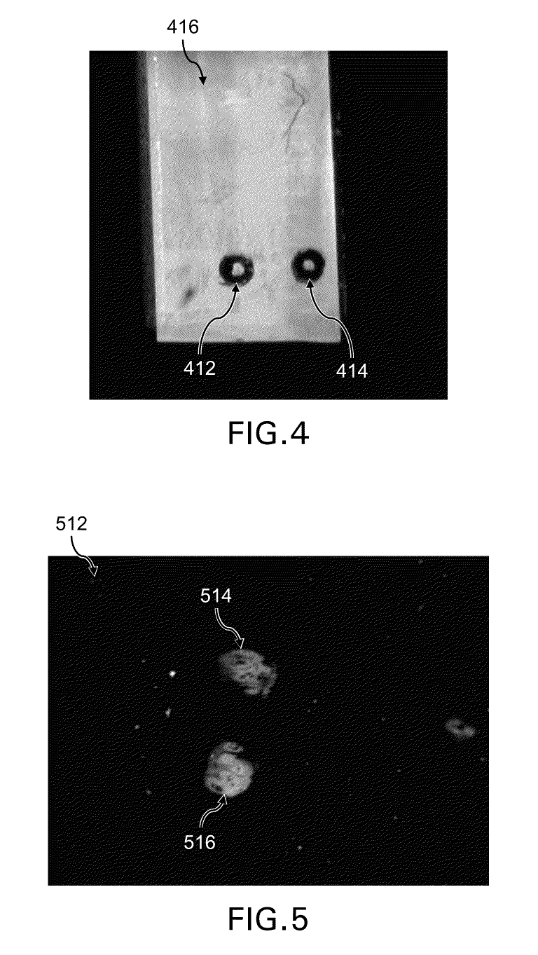

FIG. 4 is an optical diffraction image of two dark circles 412 and 414 printed (by hand) from a pipette tip onto a molded PDMS diffraction grating 416. PDMS diffraction grating 416 has a green color which indicates the optical diffraction and depends on the illumination angle. Dark circles 412 and 414 are the two different inks. FIG. 5 is an image of optical diffraction from flat glass surface 512 that was patterned by printing two different lipid inks from a PDMS grating, such as PDMS diffraction grating 416, onto flat glass surface 512. Spots 514 and 516 have different colors that relate to the angle of illumination.

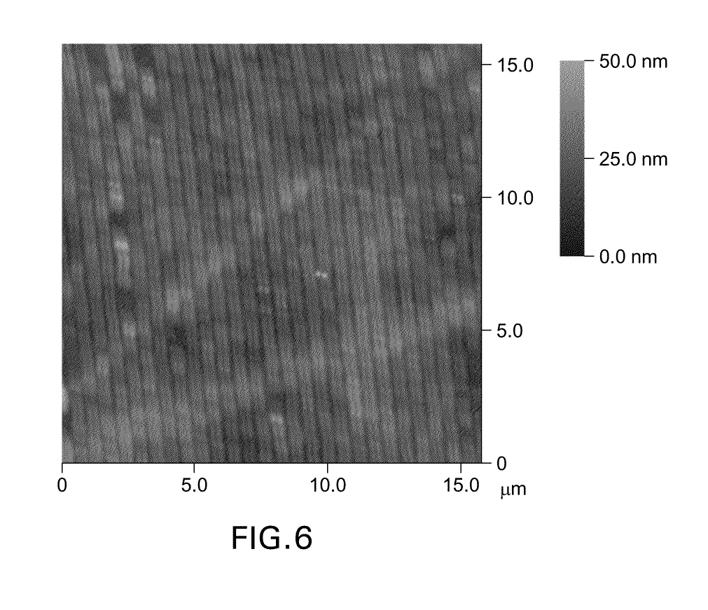

FIG. 6 is an atomic force microscopy (AFM) topographical image of lipid gratings printed using a topographically structured stamp onto a polystyrene surface (Petri dish) according to one embodiment of the present invention.

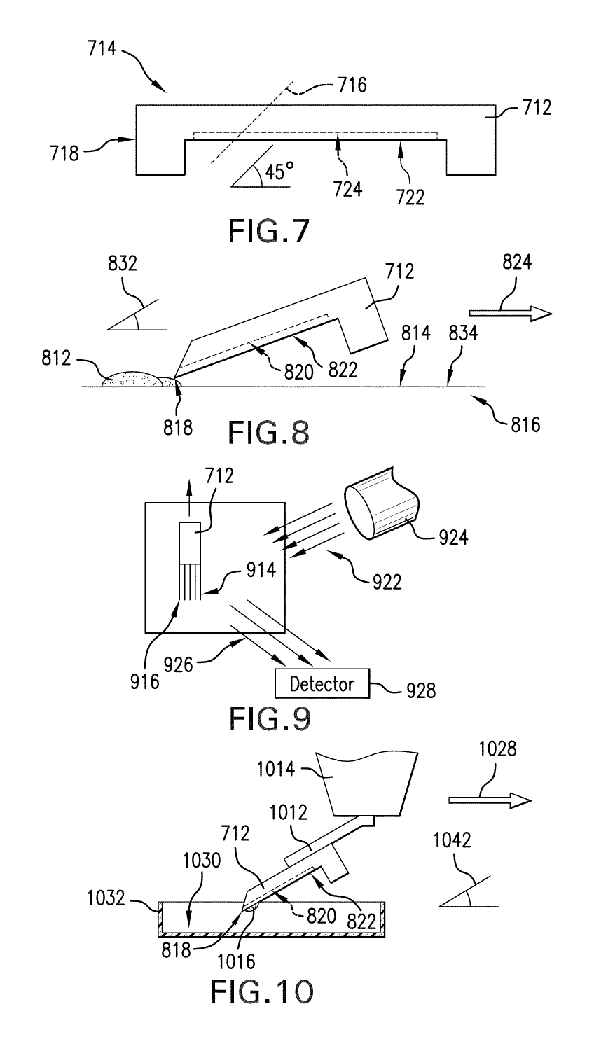

FIG. 7 shows a PDMS brush 712 being made by cutting a PDMS grating stamp 714 at a 45 degree angle at a cut 716 (indicated by dashed line) at an end 718. PDMS grating stamp 712 has a lower surface 722 including grooves 724, shown by shadow lines in FIG. 7.

FIG. 8 shows PDMS brush 712 being used to spread an iridescent lipid ink 812 on a lower surface 814 of a substrate 816 using a lower edge 818 and grooves 820 of a lower surface 822. Lower edge 818 includes grooves 820. An arrow 824 shows the direction of movement of PDMS brush 712 and the spreading of iridescent lipid ink 812. PDMS brush 712 is at an angle 832 of less than 90° with respect to substrate 816 for a portion 834 of surface 814 on which iridescent lipid ink 812 is to be spread. Lower edge 818, grooves 820 and lower surface 822 are formed by cutting PDMS grating stamp 714 in FIG. 7.

FIG. 9 shows PDMS brush 712 having spread iridescent lipid ink 812 to form a lipid multilayer grating 914 comprising lines 916 of lipid ink 812. The action of grooves 820 on lipid ink 812 forms lines 916.

Light 922 from a light source 924 that shines on lipid multilayer grating 914 is scattered as scattered light 926 and detected by a detector 928.

According to one embodiment of the present invention, a brush of the present invention may be inked and dragged along a surface with a motorized stage in order to paint form lipid multilayer structures on the surface. FIG. 10 shows PDMS brush 712 attached to a tip holder 1012 of a DPN machine 1014. A lipid ink 1016 in grooves 820 of lower surface 822 of PDMS brush 712. DPN machine 1014 is used as a brush positioning device that moves PDMS brush 712 in a direction shown by arrow 1028 to thereby spread lipid ink 1016 on a surface 1030 of a polystyrene Petri dish 1032 to form a lipid multilayer grating (not shown in FIG. 10). PDMS brush 712 is at an angle 1042 of less than 90° with respect to surface 1030 of polystyrene Petri dish 1032.

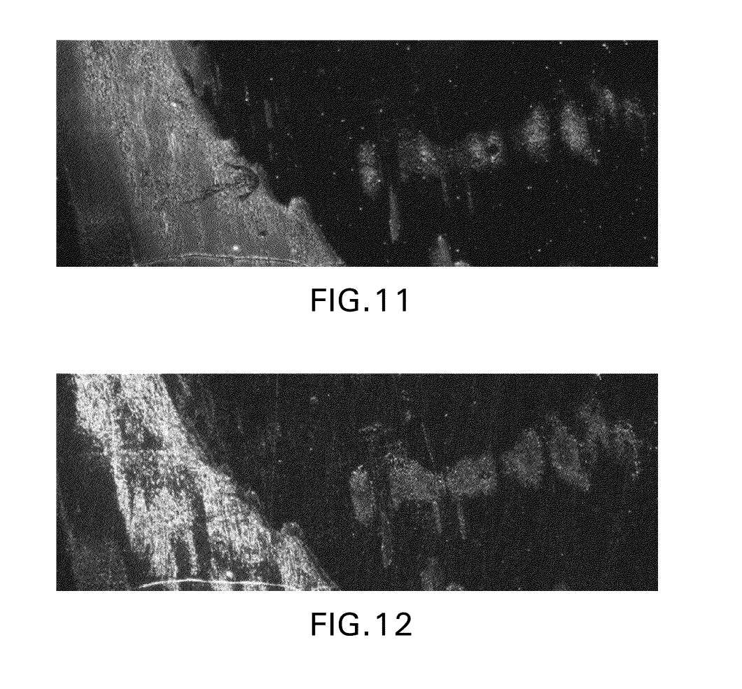

FIGS. 11 and 12 are images of light scattered from a flat surface that is patterned with lipid multilayers by dragging the lipid inked brush according to one embodiment of the present invention along the surface in the direction of the grating lines (i.e., brushing). Photos taken with illumination at low (FIG. 11) and high (FIG. 12) angles show iridescent areas of the surface. Solutions of the DOPC lipids dissolved in chloroform and deposited onto a glass slide which functioned as an ink palette. The chloroform was allowed to evaporate and then a PDMS diffraction grating was inked in a fashion similar to the way topographically structured stamp 108 is inked in FIG. 1.

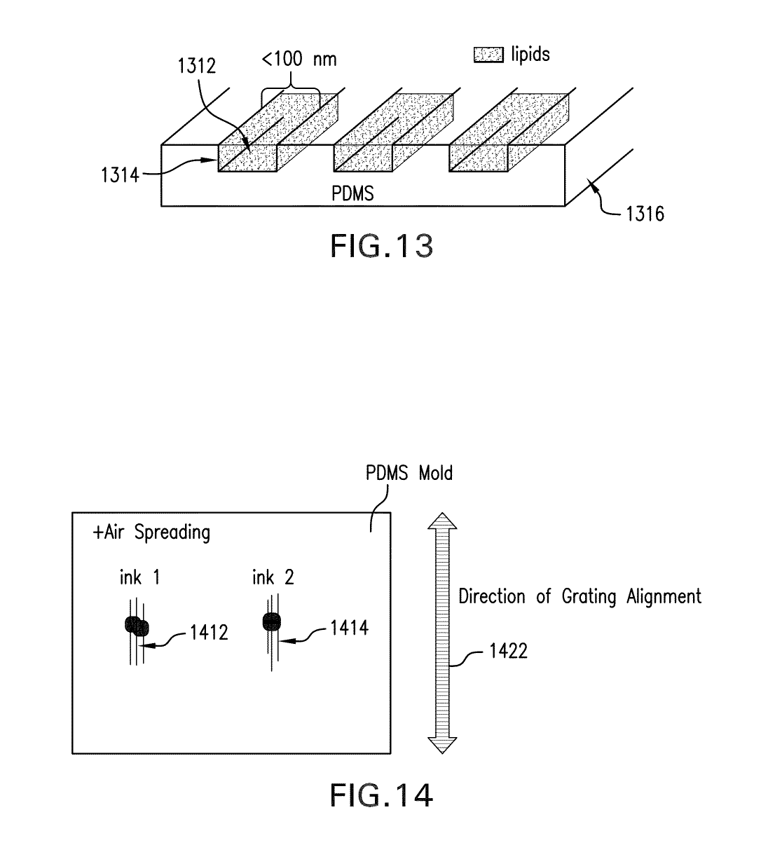

FIGS. 13 and 14 illustrate lipid spreading in air at high humidity conditions on PDMS molds. FIG. 13 shows lipid inks 1312 spreading in grooves 1314 of a PDMS stamp 1316. Grooves 1314 are <100 nm in width. FIG. 14 shows lipid inks 1412 and 1414 spreading in the direction of grating alignment, the direction of the grooves in the stamp, shown by double-headed arrow 1422.

The humidity necessary to provide good spreading of the lipid ink in the grooves depends on the particular lipid, but is generally the humidity at which the lipid has a hydration induced phase transition from a liquid to a gel state. For many lipids, a relative humidity of 40% or greater is sufficient to provide good spreading in the grooves.

Lipids inks may be made by dissolving 5 g of 1,2-dioleoyl-sn-glycero-3-phosphocholine (DOPC) in 1 L of chloroform. Once the chloroform has evaporated, the ink is kept in a vacuum chamber for at least 2 hours before use.

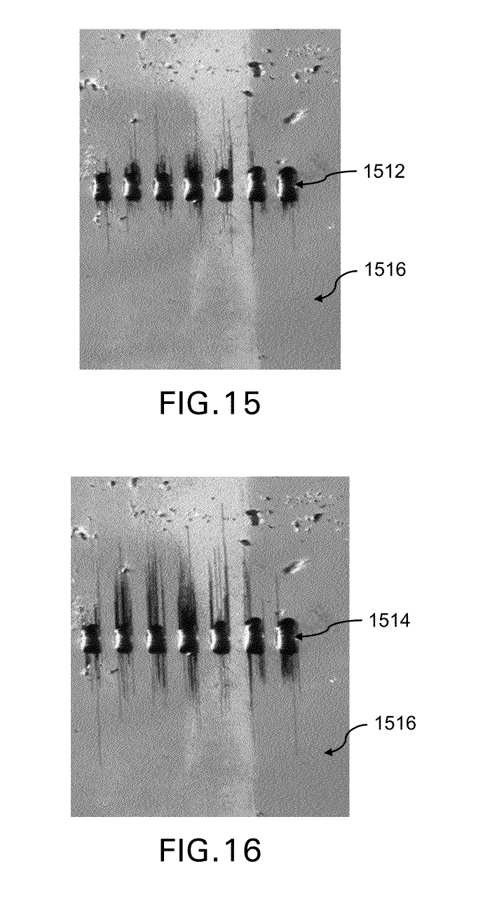

FIGS. 15 and 16 are images of light diffracted from gratings with lipid spots 1512 and 1514, respectively spreading on the surface of a PDMS mold 1516 after 5 minutes (FIGS. 15) and 20 minutes (FIG. 16) of printing using DPN at high humidity. In FIG. 16, the lipids have spread further than on the left, and the dark contrast indicates they have filled the diffraction grating lines on the surface. Lipids inks may be made by dissolving 5 g of DOPC in 1 L of chloroform and deposited in an inkwell. Once the chloroform has evaporated, the inkwell is kept in a vacuum chamber for at least 2 hours before use. To create the pattern in the pictures above, F-type cantilever tips (purchased from NanoInk, Inc.) were used to transfer the lipid ink from the inkwells to the PDMS surface using a DPN technique. This was accomplished with the NLP2000 DPN machine (NanoInk, Inc.). Once the lipids had been printed, the sample was transferred to a light microscope to obtain the images shown. Lipids were able to spread in a humidity chamber to represent high humidity.

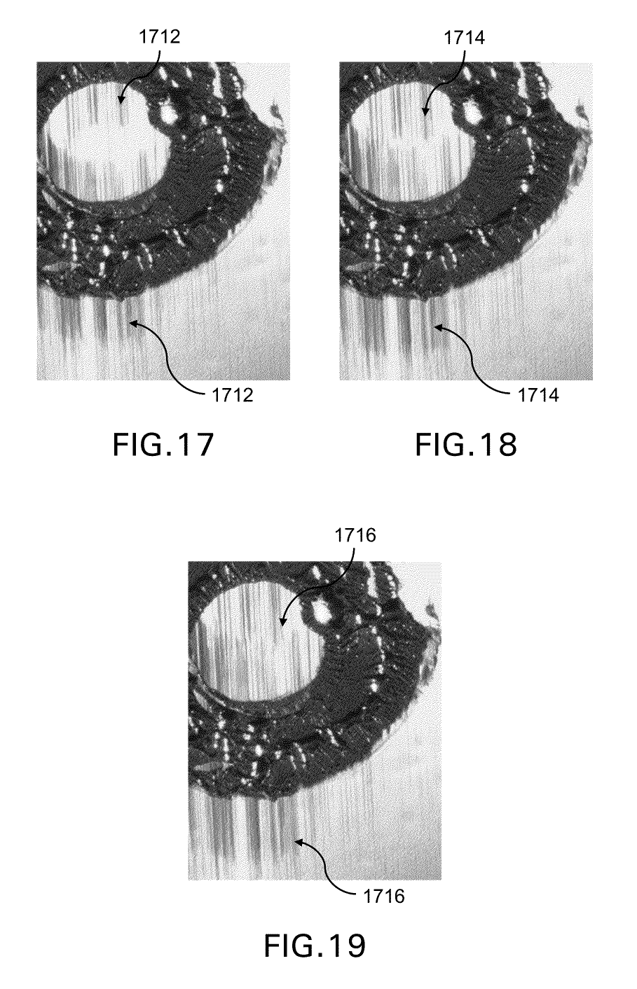

FIGS. 17, 18 and 19 are images of scattered light from a PDMS grating that was inked using a plastic pipette tip. Lipid spreads 1712, 1714 and 1716 show lipid spread after printing by micropette contact after 20 seconds (FIG. 17), 350 seconds (FIG. 18), and 500 seconds (FIG. 19) in a humidity chamber (high humidity). The pipette tip coated with phospholipids by dipping them into a chloroform solution of the lipids and allowing the chloroform to evaporate. The tip was brought in contact with the stamp by hand, and the lipids transferred to the substrate. The diameter of the ring is several millimeters, and these images were taken with a 4× magnification objective.

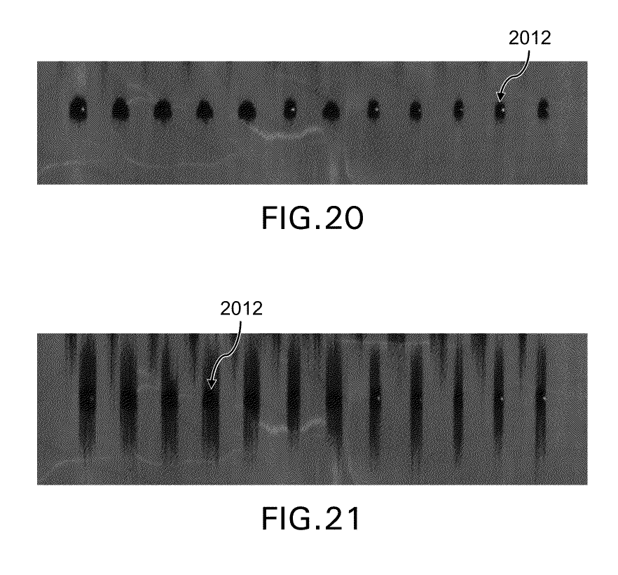

FIGS. 20 and 21 are images of scattered light from a PDMS grating with lipids 2012 deposited by DPN before (FIG. 20) and after (FIG. 21) exposure to high humidity. The spreading of lipids 2012 can be seen as a darkening of the diffraction, indicating that the lipids are filling in the grooves in the diffraction grating.

In one embodiment of the present invention, microarray technology may be combined with lipid multilayer stamping to integrate 100 different lipid formulations onto one cm².

Incorporation of functional materials such as biotinylated lipids into the gratings allows them to be used as label-free biosensors when the intensity of diffracted light is monitored as a function of time during protein binding. For example, biotinylated lipids developed for liposomal applications may be used to bind the protein streptavidin. When the protein analyte binds to these lipid multilayer grating, shape changes occur as a result of their fluidity. The sensor may also detect histidine tagged GFP when it was functionalized with nickel-chelating lipids, see M. Schelb, C. Vannahme, A. Welle, S. Lenhert, B. Ross, T. Mappes, Fluorescence excitation on monolithically integrated all-polymer chips, J. Biomed. Opt. 15, 041517-041511-041515 (2010). The sensing mechanism can be understood in terms of physical adhesion based on the interfacial energies of the solid-water, solid-oil, and oil-water interfaces, respectively. A change in any of these interfacial energies results in a change in the lipid multilayer grating height, which can be detected optically.

Lipid multilayer microarrays have recently been shown to have potential as a new technology for drug screening. In this approach, lipid-encapsulated drugs are arrayed on a surface, cells are cultured over them, and assays for drug efficacy are carried out in a microarray format. The multilayer patterns may be formed by DPN, may have subcellular dimensions to allow cell adhesion to the substrate, and may be of controllable thickness to allow drug encapsulation. Also, different dosages of drugs may be delivered from different areas of the array.

In one embodiment, the present invention provides a method that combines the lateral patterning capabilities and scalability of microcontact printing with the topographical control of nanoimprint lithography and the multimaterial integration aspects of dip-pen nanolithography in order to create nanostructured lipid multilayer arrays. This approach is denoted multilayer stamping. The distinguishing characteristic of this method is that it allows control of the lipid multilayer thickness, which is a crucial nanoscale dimension that determines the optical properties of lipid multilayer nanostructures. The ability to integrate multiple lipid materials on the same surface is also demonstrated by multi-ink spotting onto a PDMS stamp, as well as higher-throughput patterning (on the order of 2 cm² s⁻¹ for grating fabrication) and the ability to pattern lipid materials that could not previously be patterned with high resolution by lipid DPN, for example, the gel-phase phospholipid 1,2-dipalmitoyl-sn-glycero-3-phosphocholine (DPPC) or the steroid cholesterol.

Lipid bilayers and multilayers play an important role in nature by mediating ubiquitous functions in all living organisms and have applications in massive parallel sensing of biological agents (e.g., receptor-mediated signaling),¹ energy conversion and storage (cellular respiration),² and delivery of materials throughout cells, organisms, and ecosystems (molecular transport).³ Furthermore, a variety of niche applications of lipids in nature demonstrate lipid multilayer nanostructure-function relationships. For example, rapidly adaptive camouflage or color change in cephalopods is made possible by Bragg reflection from regularly stacked refractive protein layers organized and regulated at nanometer scales by lipid membranes (iridophores).⁴ The ability to reconstruct such biologically inspired lipid nano- and microstructures synthetically has promising implications in both biology and nanotechnology. Supported lipid bilayers are well-established as model membrane systems and have been patterned by a variety of methods, including microcontact printing.⁵ Bulk lipid multilayers can also be formed on surfaces and are widely used for NMR-based structural studies of reconstituted transmembrane proteins.⁶

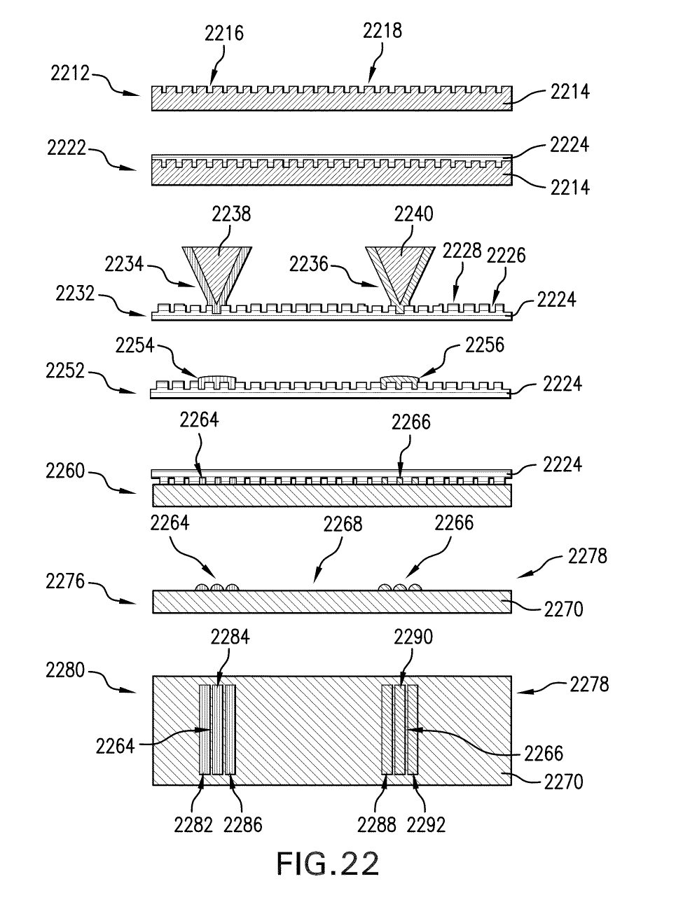

FIG. 22 shows a multilayer stamping process of the present invention used in this example. At step 2212 a master 2214 of desired dimensions is provided. Master 2214 has grooves 2216 and ridges 2218. Silicon master 2214 is used in step 2222 to create a topographically structured stamp 2224 having grooves 2226 and ridges 2228. In step 2232 lipid inks 2234 and 2236 are spotted on topographically structured stamp 2224 using respective dip pens 2238 and 2240 using DPN. In step 2252 inked topographically structured stamp 2224 having deposited lipid inks 2254 and 2256 is incubated in a humidity chamber (not shown) having a relative humidity greater than 95% humidity, during which lipid inks 2254 and 2256 spread longitudinally (away and towards the viewer in FIG. 22) inside grooves 2226 of topographically structured stamp 2224 to more evenly fill grooves 2226. In step 2260, diffraction gratings 2264 and 2266, made of lipid inks 2254 and 2256, respectively, are printed on a surface 2268 of a substrate 2270 by placing inked stamp 2224 in contact with surface 2268. In step 2276, topographically structured stamp 2224 is removed leaving patterned arrays 2264 and 2266 on substrate 2268, thereby forming patterned substrate 2278. View 2280 is a top view of patterned substrate 2280. Patterned array 2264 comprises lipid multilayer structures 2282, 2284 and 2286 made from lipid ink 2254. Patterned array 2266 comprises lipid multilayer structures 2288, 2290 and 2292 made from lipid ink 2256. Patterned array 2264 and 2264 are each diffraction gratings.

An important difference between the process shown in FIG. 22 and traditional microcontact printing is that the result is a multilayer pattern in which the three dimensional topography is controlled by the stamp topography. The ability to control the thickness of lipid multilayers between 1 and 100 nm (and above) is important to the size dependant function of lipid multilayers. In addition, when DPN is used to deposit lipid inks on the stamp, the site-specific material-deposition capabilities of DPN may be used to allow multiplexed patterning. Although the DPN tip has a radius on the order of 50 nm, when fluid lipid inks are used, a DPN tip is capable of depositing much larger spots, up to several micrometers wide and thick.²⁴ After deposition, fluid lipid inks spontaneously spread at high relative humidity (RH), but, because they are confined in the grating channels, the lipids spread only longitudinally and not laterally. This anisotropic spreading is caused by contact line pinning at the edges of the topographical structures.²⁵ In some embodiments of the present invention, the topographical structures of the stamp allows both lateral and longitudinal control of grating feature size.

Because lipid inks in FIG. 22 are different lipid inks, the method shown in FIG. 22 shows the simultaneous patterning of two different lipid inks. The method shown in FIG. 22 may also be used to print three or more patterns of lipid inks simultaneously.

Although gratings comprising parallel lines are shown being printed in FIG. 22, the method shown in FIG. 22 may be used to print patterns having various shapes and arrangements.

In one embodiment of the present invention, the master may be made of silicon.

In one embodiment, the stamp may be made of polydimethoxysilane (PDMS).

Although a DPN technique is shown in FIG. 22, other spotting methods may be employed in various embodiments of the present invention. For example, a pipette tip may be used to deposit or spot lipid inks on the stamp.

The dimensions of the lipid multilayer gratings shown in FIG. 22 are determined by the stamp-groove height and pitch.

The substrate used in FIG. 22 may be made of glass, silicon, polymers or other materials.

The lipid inks and lipid multilayer structures of the present invention may include dyes such as fluorescent dyes. Examples of suitable fluorescent dyes include various fluorescent organic molecules, fluorescent proteins, pigments, nanoparticles, etc.

### EXAMPLES

### Example 1

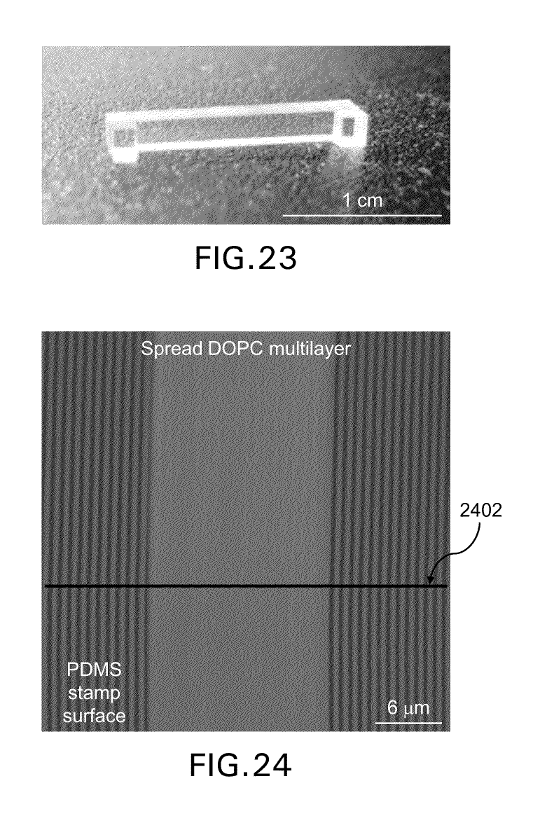

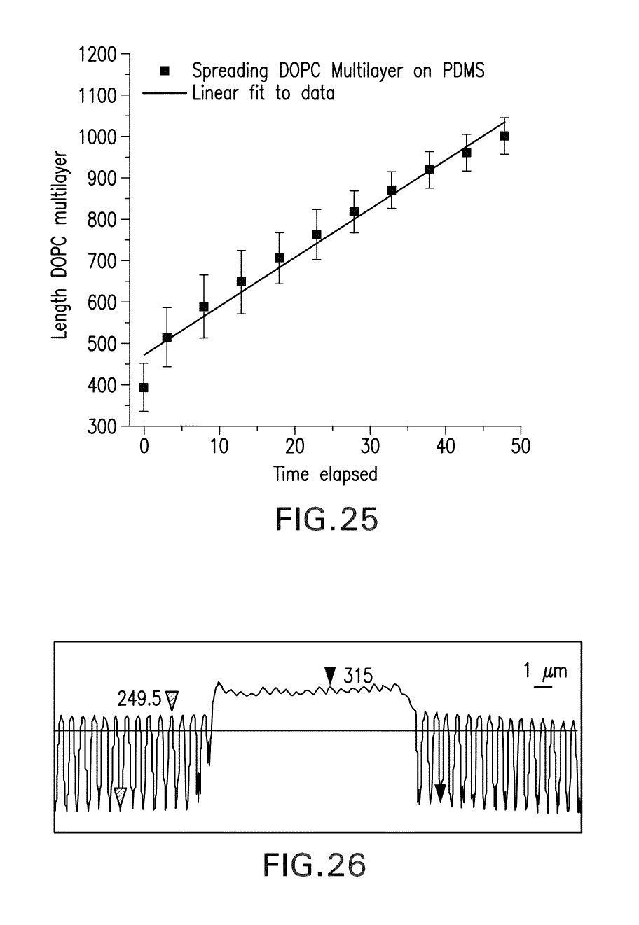

Method of making a PDMS grating stamp. The initial step in this fabrication approach involves making a PDMS grating stamp, shown in FIG. 23, from a silicon grating master (not shown). The PDMS grating stamp of FIG. 23 is created by pouring of PDMS over the silicon master. The PDMS grating stamp was created from a silicon grating master (20 mm×9 mm) of 250 nm height and 700 nm pitch. FIGS. 24, 25 and 26 show characterization of a PDMS stamp and a lipid (1,2-dioleoyl-sn-glycero-3-phosphocholine, DOPC) coating according to one embodiment of the present invention. To ensure that the PDMS stamp reproduces the silicon gratings faithfully, the surface of the stamp is characterized with atomic-force microscopy (AFM). The PDMS stamp is then inked with lipids deposited with an array of DPN tips used as a pin spotter to deposit one large (˜10 μm×10 μm×10 μm) lipid droplet. The stamp is then immediately placed in a closed Petri dish at >95% RH for ˜1 h. This procedure allows the lipid (DOPC) to spread longitudinally in the stamp grooves to give a uniform height (layer) on the stamp before t-CP on the surface as shown in FIG. 24. FIG. 24 is an AFM height image of the spread DOPC multilayer film deposited on the PDMS stamp by DPN. Fluid lipids readily spread at high humidity on hydrophobic surfaces such as PDMS,^{7a} but such multilayer spreading behavior of phospholipids has not been quantitatively characterized. The spreading rate of DOPC on the PDMS stamp was tracked by capture of time-lapse fluorescence images. FIG. 25 shows the linear progress of DOPC multilayers along the PDMS stamp grooves. As shown in FIG. 25, the DOPC spreads vertically along the PDMS stamp grooves, and the rate of DOPC spreading on the PDMS stamp was measured to be linear with a spreading rate of ˜12 μm per min. The error bars represent measurements from four different spread DOPC multilayers. The stamp grooves limit the lateral spreading of phospholipids, and the linear multilayer spreading rate of DOPC during the first 50 minutes of exposure to high humidity, measured from the slope of FIG. 25, was ˜12 μm min⁻¹. FIG. 26 shows a height of 65 nm of the DOPC coating on the PDMS stamp. FIG. 26 shows a line trace of a (region) line 2402 in FIG. 24 showing the 250 nm height of the PDMS stamp grooves together with the 65 nm height of DOPC multilayer deposited by DPN.

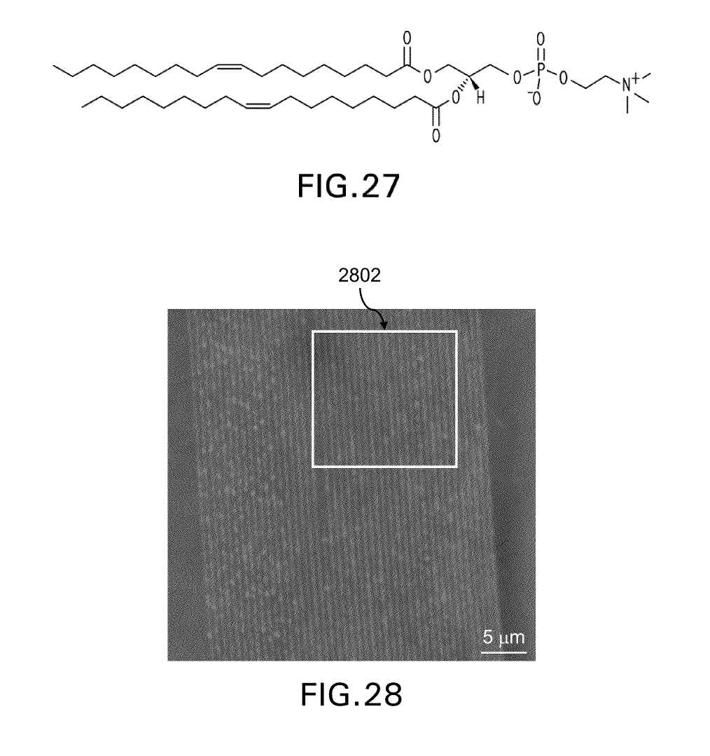

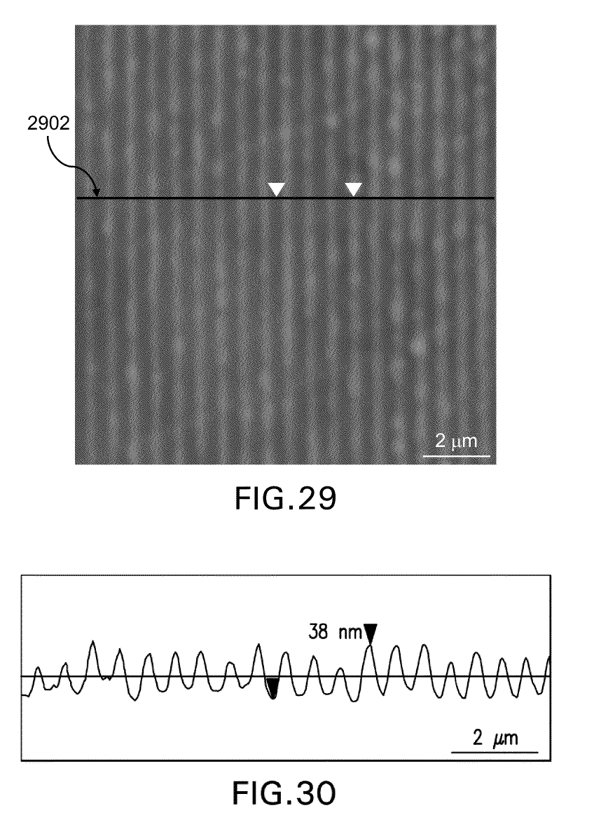

Lipid multilayer gratings composed of DOPC. FIG. 27 shows the structure of DOPC. FIG. 28 is an AFM height image of 22 μm wide DOPC diffraction grating stamped with the inked PDMS stamp on a polystyrene (PS) surface. FIG. 29 is a close-up view of boxed region 2802 of FIG. 28 showing continuous DOPC lines spaced 555 nm apart that function as diffraction gratings. The lines cover a length of ˜1 mm in the vertical direction. FIG. 30 is a line trace of a line (region) 2902 in FIG. 28 showing the similar height (38±9 nm) of the DOPC features created with different PDMS stamps.

FIGS. 28 and 29 show the DOPC diffraction grating elements after they have been stamped on a polystyrene (PS) surface. FIGS. 28 and 29 show that continuous and distinct grating elements of controlled dimensions can be created using the techniques of the present invention. Some evidence indicates phospholipid dewetting from the surface as shown by the formation of droplets from the grating lines.⁸ The grating elements created by this method diffract light and have an aspect ratio (grating height/grating pitch ˜0.1) which is similar to that of features made by DPN.^{7a} The stamping process step takes less than 5 s to complete, as compared with the approximately 30 min needed for a single DPN tip to make a grating over an area such as that shown in FIG. 28. In the stamping approach presented here, we can deposit multiple inks side by side on the PDMS stamp by DPN and obtain various diffraction colors from those inks by illuminating them at different angles.

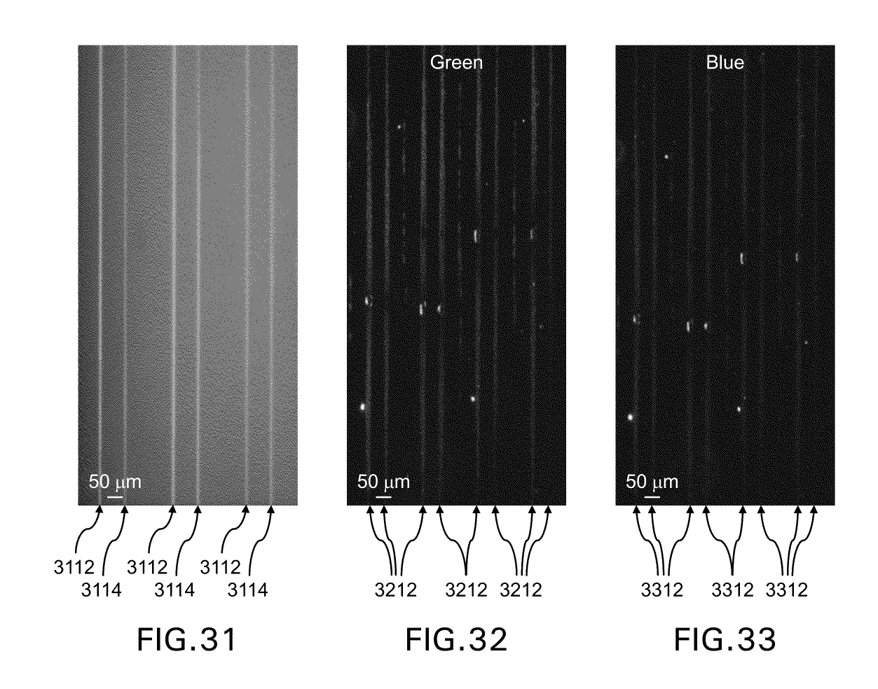

The multifunctionality permitted by techniques of the present invention allows multiple chemical functionalities to be integrated on the same surface in combination with nanostructure-dependent optical properties such as iridescence. FIG. 31 is a fluorescence microscopy image of two lipid inks patterned by DPN on a PDMS stamp according to one embodiment of the present invention. FIG. 31 shows part of the inked PDMS stamp surface (before stamping) with red and green fluorescently labeled DOPC inks on a 140-nm stamp (555 nm pitch), which was used to create diffraction gratings on the surface. Two different inks were created with rhodamine B (red) and 1,2-dioleoyl-sn-glycero-3-phosphoethanolamine-N-5-dimethylamino-1-naphthalenesulfonyl (green) dyes, and DPN was performed on the PDMS surface with six different cantilevers. The inks were then allowed to spread on the stamp, producing an alternate set of three green and three red vertical lines. FIG. 31 shows that two different inks can be patterned side by side. The vertical green lines and red lines, indicated by arrows 3112 and 3114, respectively, represent two different DOPC inks that have spread on a PDMS stamp (140 nm groove height, 555 nm pitch).

FIGS. 32 and 33 are bright-field diffraction images of two different lipids stamped on a PS surface (4× magnification). FIGS. 32 and 33 show that two different colors can be observed: green, shown by lines 3212 in FIG. 32, and blue, shown by lines 3312 in FIG. 33). The different colors are obtained when the angle of incident light is changed. The green color was seen at ˜70° of white incident light with the surface normal, whereas the blue color was obtained at an incident angle of ˜58°. FIGS. 32 and 33 show the different diffraction colors obtained by stamping the multi-ink gratings on a polystyrene surface. The colors correspond to the wavelength of light diffracted according to the grating equation:

d(sin θₘ+sin θᵢ)=nλ (1)

where d is the period of the grating, θₘ and θᵢ are the angles of diffraction maxima and incidence respectively, n is the diffraction order, and is the wavelength of light. In our setup we use white incident light and observe the intensity of light at θₘ˜0° normal to the grating plane. The color observed by a color camera depends only on the grating period and θᵢ, which can be adjusted according to the stamp topography (period of 555 nm) illuminated at θᵢ=58° (for blue color). The vertical length of the DOPC patterns was >1 mm, and the area covered with a single stamping was ˜0.5 mm². A grating area of at least 2.5 mm² can therefore be created by stamping of a single inked stamp over the course of five successive attempts. The eight vertical lines of blue and green were obtained by simultaneous DOPC DPN on a PDMS stamp with eight different cantilevers arranged parallel in an array. Theoretically, increasing the number of simultaneous DPN cantilevers will result in a greater stamp (surface) coverage with features that diffract light. Furthermore, DPN is not the only method that can be used to ink the PDMS stamp; other scalable microarray techniques like pin-spotting²⁶ and inkjet printing²⁷ can also be used. In the work reported here, we have used DPN as a “pin-spotter” to demonstrate that this approach to inking PDMS stamps can result in diffraction gratings. The cantilevers may also be coated with inks other than DOPC to create multiplexed diffraction gratings over a large area. Increasing the size of the PDMS stamp will also lead to higher surface coverage.

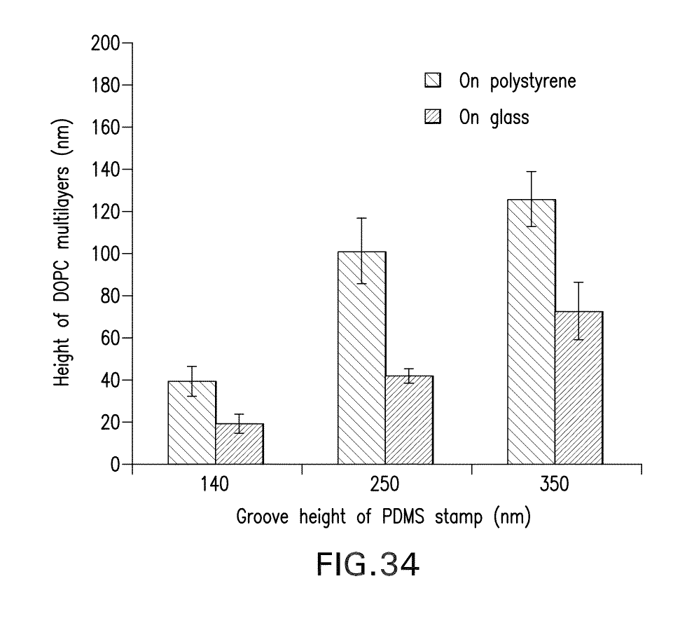

FIG. 34 is a graph showing control over lipid multilayer height on different surfaces—polystyrene and freshly cleaned glass—with different PDMS stamps. The PDMS stamps were created from silicon grating masters of different height and varying groove depth: 140, 250, and 350 nm (700 nm pitch). The error bars indicate standard deviations of measurements made by AFM. To show that DOPC features of controlled dimensions can be fabricated, the lipid DOPC was stamped on two different surfaces, polystyrene (PS) and glass, using three different stamp dimensions as shown in FIG. 34. The final feature height obtained by this method is ˜40% of the groove height of the PDMS stamp (silicon master) chosen on PS and ˜25% of it on glass. The DOPC grating height on PS was slightly greater than that on glass, and we attribute this difference to the variation in the initial DOPC height on the PDMS stamp, different surface energies, and an inevitable variation in the stamping force. Qualitatively, the lipid multilayers are observed to be more stable on a PS surface than on a glass slide—lipid dewetting instabilities can occur within hours on glass with exposure to ambient RH. We attribute this substrate dependence to the different surface energies of PS and glass;^{28,29} as PS is more hydrophobic than glass. Other potential limitations that affect the lateral resolution of stamped features is the stamp deformation during stamp removal from the silicon master and during contact with the substrate.³⁰ In comparison, the traditional DPN method also suffers from limitations, i.e., slow throughput and ink depletion from the tip,³¹ which affect pattern fidelity (reproducibility), especially important for features like diffraction gratings, which require precise control over the features aspect ratios. Our method also suffers ink depletion from the stamp, but we have found that each stamp can be used for at least five successive stamping attempts before the features start to undergo loss of uniformity. Further, improvement of throughput of the stamping device might include a roll-on stamp device,³² and multilayer heights might be further controlled by mechanical control of the lipid stamping force.^{30b}

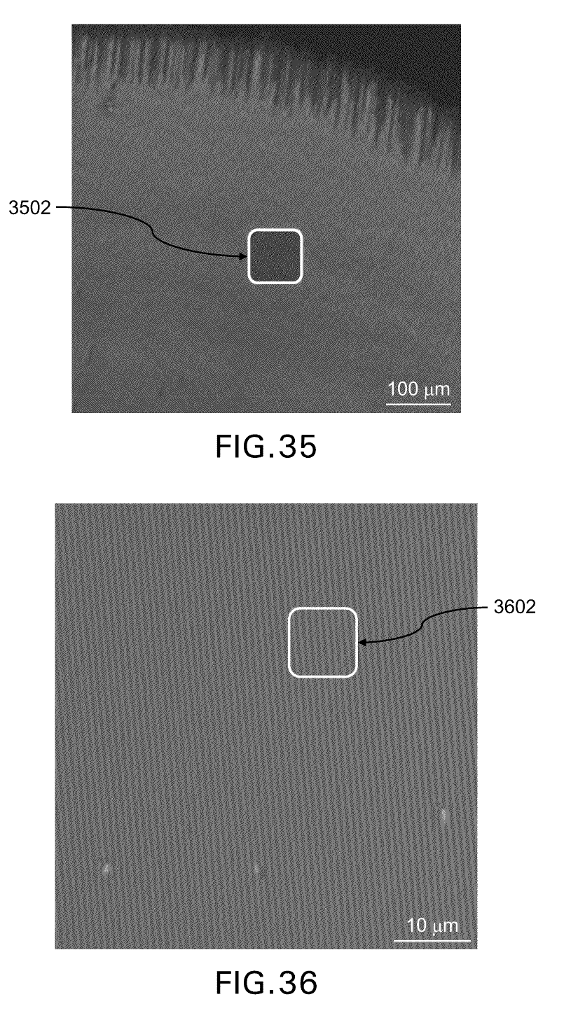

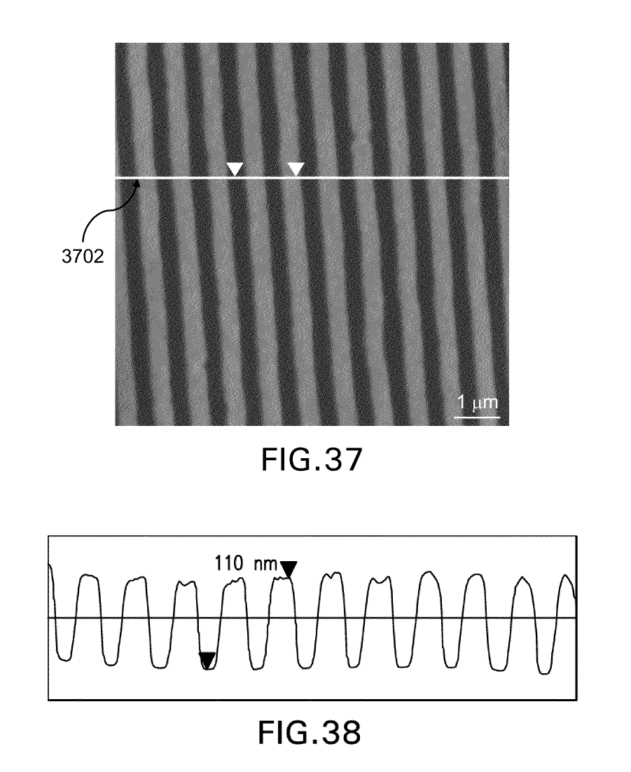

Lipid multilayer gratings formed with the gel-phase lipid DPPC. Gratings may also be created with other lipids besides fluid DOPC. FIGS. 35, 36 and 37 show green diffraction together with the corresponding AFM image of the stamped grating structures of a gel-phase phospholipid like 1,2-dipalmitoyl-sn-glycero-3-phosphocholine (DPPC), which cannot be patterned by DPN at room temperature, as it is not fluid at room temperature (Tm=41° C.). The gratings gave three distinct diffraction colors (red, green, and blue) at different angles of incident light. Importantly, these DPPC gratings can be immersed in water under ambient conditions (humidity up to 60%), which is a significant practical advantage over DOPC-based lipid multilayer gratings which require that immersion in water be carried out in a dehydrating atmosphere, such as pure nitrogen.^{7a} This technique may also be used to create diffraction gratings with lipids that are not phospholipids, in particular those that cannot be patterned by DPN or techniques based on spin-coating multilayers.³³ The steroid cholesterol was used for this purpose, as it is a fundamentally different type of biological lipid yet is still an integral component of animal cell membranes. FIG. 35 is red diffraction obtained from stamped DPPC gratings with a 140 nm tall and 700 nm pitch stamp. FIG. 36 is an optical micrograph with surface-enhanced ellipsometric contrast (SEEC) imaging of a white square region 3502 in FIG. 35 showing DPPC grating lines over a large area. FIG. 37 is an AFM height image of a white square region 3602 in FIG. 36. FIG. 38 is a line trace along line 3702 of gratings in FIG. 37 showing an average height of 110 nm±10 nm. The DPPC gratings are stamped onto a commercially available silicon oxide surface (Surf) for greater optical contrast.

Experimental Details. Creation of μ-CP Stamps: PDMS μ-CP stamps were created from silicon masters with the desired pitch and groove height purchased directly from LightSmyth Technologies (Eugene, Oreg.). The silicon masters were initially cleaned with piranha solution and later passivated with a 0.2% (by volume) octadecyltrichlorosilane solution in toluene. The PDMS stamp of desired dimensions was prepared from a Sylgard 184 (Dow Corning, Midland, Mich.) elastomer gel poured over the passivated silicon master and cured overnight at 65° C. DPN was then used to deposit the phospholid ink on the structured PDMS stamp by means of a NLP 2000 lithography system and M-type cantilevers (NanoInk, Skokie, Ill.).

Phospholipid Tip Inking and Spreading: DOPC (20 g L⁻¹ solution in chloroform), DPPC (10 g L⁻¹ solution in chloroform), 1,2-dioleoyl-sn-glycero-3-phosphoethanolamine-N-lissamine rhodamine B sulfonyl (DOPE-RB, 1 mol %, 1 g L⁻¹ red dye solution in chloroform), and 1,2-dioleoyl-sn-glycero-3-phosphoethanolamine-N-5-dimethylamino-1-naphthalenesulfonyl (1 mol %, 1 g L⁻¹ green dye solution in chloroform) were purchased from Avanti Polar Lipids (Alabaster, Ala.) and used as received. Custom inkwell microchannels were used to coat the M-type cantilever tips (NanoInk) during the DPN step. The inkwell was kept under vacuum overnight so that the chloroform would evaporate. The tips were dipped in the microwells at a relative humidity (RH) of ˜75% for 5 min to receive a uniform coating of lipids. RH was automatically controlled by means of a combination of water bath and nitrogen circulation in the RH-control chamber. The inks were kept in closed tins that prevented their exposure to external light sources. After tip inking, the coated tip was placed in contact with the μ-CP stamp and the deposited lipid was allowed to spread under high humidity (>95%). The spread lipid stamp channels were then used to create diffraction gratings.

Multilayer Stamping: For DOPC stamping on the PS or silicon oxide surfaces, the PDMS stamp was inked as described above, and placed in contact with the substrate. Slight pressure was then applied to the stamp for the purpose of adequate printing. In the case of DPPC, 1 μL of a 10 g L⁻¹ chloroform solution of DPPC was spotted on a 140 nm tall PDMS stamp surface, and either allowed to dry in a vacuum for at least 1 h, or left to dry in air for ˜45 seconds until slightly moist with chloroform (the condition leading to the most uniform gratings), and subsequently stamped onto a silicon oxide surface. The stamps were left in direct contact with the surface for ˜0 seconds before careful removal of the stamp.

Surfaces Used and Sample Preparation: The diffraction gratings were created by multilayer stamping of the inked PDMS stamps on PS, glass, and Sarfus surfaces. Tissue-culture grade PS Petri dishes (#82050-546) and glass slides (#48366-227) were purchased from VWR (West Chester, Pa.). PS dishes were used as received and cut before patterning for ease of AFM imaging. Glass slides were freshly cleaned with a 5:1:1 (by volume) H₂O:H₂O₂:NH₄OH solution before use. The Sarfus surface was provided by Nanolane (Montfort-le-Gesnois, France) and was freshly prepared for stamping by removal of the top protective film.

Characterization and Imaging Techniques: A Ti-E epifluorescence inverted microscope (Nikon Instruments, Melville, N.Y.) fitted with a Retiga SRV (QImaging, Canada) CCD camera (1.4 MP, Peltier cooled to −45° C.) was used for fluorescence and brightfield imaging of the lipid gratings on PS and glass surfaces. The same setup was used to capture diffraction images in bright-field mode with a fiber-optic white light source (Eco Light 150, MK Photonics, Albuquerque, N. Mex.). The various colors of diffraction were produced by different angles of incident light (fiber-optic guide) on the surface.

After fluorescence-microscope imaging, the patterns were imaged in tapping mode with a Dimension 3000 AFM (Veeco Instruments, Plainview, N.Y.) and tapping mode AFM cantilevers (# OMCLAC160TS-W2, 7 nm nominal tip radius, 15 μm tip height, 42 N m⁻¹ spring constant, Olympus, Center Valley, PA). Noncontact mode AFM imaging is suitable for imaging micro- and nanoscopic fluid droplets.³⁴ Tip-sample interaction forces were kept at a minimum to prevent sample deformation and adhesion of the fluid lipid multilayers to the tip. SEEC³⁵ microscopy was used in DIC mode with an upright microscope AxioImager A2M in reflection mode (Zeiss, Göttingen, Germany) fitted with a HITACHI HV-F22GV (HITACHI, Japan) 3 CCD camera (1.4 MP). This technique is based on the use, as substrates, of a new generation of microscope slides (Surfs) that allow the strong enhancement of the sample contrast with a conventional optical microscope. All experiments were performed at ambient temperature (25° C.±2%).

### Example 2

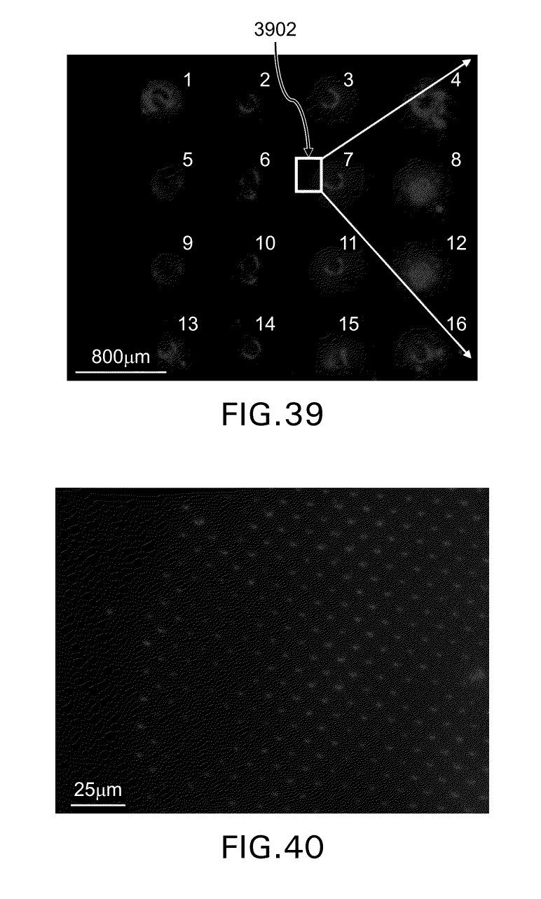

FIG. 39 shows the results of the experiment where 16 different liposomal drug formulations were arrayed onto a polydimethylsiloxane stamp and arrayed onto a glass surface. Integration of 16 different liposomal formulations of the drug valinomycin, plus a control into a lipid multilayer microarray. FIG. 39 shows a fluorescence micrograph of 16 spots printed onto a glass slide. Each spot consists of a different liposomal formulation. Each spot in FIG. 39 is numbered, and the compositions are: [1] DOTAP only, [2] DOTAP+Valinomycin (1:1), [3] DOTAP+Valinomycin (2:1), [4] DOTAP+Valinomycin (4:1), [5] DOTAP+Valinomycin (8:1), [6] DOTAP/DOPE(30:70)+Valinomycin (1:1), [7] DOTAP/DOPE(30:70)+Valinomycin (2:1), [8] DOTAP/DOPE(30:70)+Valinomycin (4:1), [9] DOTAP/DOPE(30:70)+Valinomycin (8:1), [10] DOTAP/Cholesterol(20 mol %)+Valinomycin (1:1), [11] DOTAP/Cholesterol(20 mol %)+Valinomycin (2:1), [12] DOTAP/Cholesterol(20 mol %)+Valinomycin (4:1), [13] DOTAP/Cholesterol(20 mol %)+Valinomycin (8:1), [14] DOTAP/DOPE(30:70)/Cholesterol(20 mol %)+Valinomycin (1:1), [15] DOTAP/DOPE(30:70)/Cholesterol(20 mol %)+Valinomycin (2:1), [16] DOTAP/DOPE(30:70). FIG. 40 is a high magnification of outlined part 3902 containing spot 7 in FIG. 39, showing transfer of the stamp geometry. In this case, a stamp composed of microwells was used, resulting in patterns of dots that may be an effective pattern for drug screening in cell culture.

Having described the many embodiments of the present invention in detail, it will be apparent that modifications and variations are possible without departing from the scope of the invention defined in the appended claims. Furthermore, it should be appreciated that all examples in the present disclosure, while illustrating many embodiments of the invention, are provided as nonlimiting examples and are, therefore, not to be taken as limiting the various aspects so illustrated.

While the present invention has been disclosed with references to certain embodiments, numerous modifications, alterations and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

### REFERENCES

The following references are referred to above and are incorporated herein by reference:
1. a) P. Banerjee, Indian J. Biochem. Biophys. 1993, 30, 358; b) E. A. Dennis, S. G. Rhee, M. M. Billah, Y. A. Hannun, FASEB J. 1991, 5, 2068.
2. a) L. J. Sweetlove, A. Fait, A. Nunes-Nesi, T. Williams, A. R. Fernie, Crit. Rev. Plant Sci. 2007, 26, 17; b) D. F. S. Rolfe, J. T. Wang, M. Y. Sun, J. Exp. Marine Biol. Ecol. 2010, 391, 73.
3. a) R. G. W. Anderson, K. Jacobson, Science 2002, 296, 1821; b) F. R. Maxfield, I. Tabas, Nature 2005, 438, 612.
4. a) M. Izumi, A. M. Sweeney, D. DeMartini, J. C. Weaver, M. L. Powers, A. Tao, T. V. Silvas, R. M. Kramer, W. J. Crookes-Goodson, L. M. Mathger, R. R. Naik, R. T. Hanlon, D. E. Morse, J. Royal Soc. Interface 2010, 7, 549; b) R. M. Kramer, W. J. Crookes-Goodson, R. R. Naik, Nat. Mater. 2007, 6, 533; c) L. M. Mathger, E. J. Denton, N. J. Marshall, R. T. Hanlon, J. Royal Soc. Interface 2009, 6, 5149.
5. a) J. S. Hovis, S. G. Boxer, Langmuir 2001, 17, 3400; b) S. Majd, M. Mayer, Angew. Chem. Int. Ed. 2005, 44, 6697.
6. J. J. Devries, H. J. Berendse, Nature 1969, 221, 1139.
7. a) S. Lenhert, F. Brinkmann, T. Laue, S. Walheim, C. Vannahme, S. Klinkhammer, M. Xu, S. Sekula, T. Mappes, T. Schimmel, H. Fuchs, Nat. Nanotechnol. 2010, 5, 275; b) S. Lenhert, P. Sun, Y. H. Wang, H. Fuchs, C. A. Mirkin, Small 2007, 3, 71; c) O. A. Nafday, S. Lenhert, Nanotechnol. 2011, 22; d) S. Sekula, J. Fuchs, S. Weg-Remers, P. Nagel, S. Schuppler, J. Fragala, N. Theilacker, M. Franueb, C. Wingren, P. Ellmark, C. A. K. Borrebaeck, C. A. Mirkin, H. Fuchs, S. Lenhert, Small 2008, 4, 1785.
8. L. Perino-Gallice, G. Fragneto, U. Mennicke, T. Salditt, F. Rieutord, Eur. Phys. J. E 2002, 8, 275.
9. M. Mathieu, D. Schunk, S. Franzka, C. Mayer, N. Hartmann, J. Vacuum Sci. Technol. A 2010, 28, 953.
10. O. A. Nafday, J. R. Haaheim, F. Villagran, Scanning 2009, 31, 122.
11. a) J. Haaheim, O. A. Nafday, Scanning 2008, 30, 137; b) D. S. Ginger, H. Zhang, C. A. Mirkin, Angew. Chem. Int. Ed. 2004, 43, 30; c) A. B. Braunschweig, F. W. Huo, C. A. Mirkin, Nat. Chem. 2009, 1, 353; d) K. Salaita, Y. Wang, C. A. Mirkin, Nat. Nanotechnol. 2007, 2, 145.
12. a) Y. Wang, L. R. Giam, M. Park, S. Lenhert, H. Fuchs, C. A. Mirkin, Small 2008, 4, 1666; b) S. Sekula, J. Fuchs, S. Weg-Remers, P. Nagel, S. Schuppler, J. Fragala, N. Theilacker, M. Franzreb, C. Wingren, P. Ellmark, C. A. K. Borrebaeck, C. A. Mirkin, H. Fuchs, S. Lenhert, Small 2008, 4, 1785.
13. a) S. K. Saha, M. L. Culpepper, J. Manufact. Sci. Eng.—Trans. ASME 2011, 133; b) K. Gupta, D.-H. Kim, D. Ellison, C. Smith, A. Kundu, J. Tuan, K.-Y. Suh, A. Levchenko, Lab Chip 2010, 10, 2019.
14. a) D. Qin, Y. N. Xia, G. M. Whitesides, Nat. Protocols 2010, 5, 491; b) Y. N. Xia, G. M. Whitesides, Annu. Rev. Mat. Sci. 1998, 28, 153; c) V. Linder, H. K. Wu, X. Y. Jiang, G. M. Whitesides, Analyt. Chem. 2003, 75, 2522.
15. F. W. Huo, Z. J. Zheng, G. F. Zheng, L. R. Giam, H. Zhang, C. A. Mirkin, Science 2008, 321, 1658.
16. R. S. Kane, S. Takayama, E. Ostuni, D. E. Ingber, G. M. Whitesides, Biomaterials 1999, 20, 2363.
17. M. Mrksich, G. M. Whitesides, Trends Biotechnol. 1995, 13, 228.
18. a) S. Y. Yang, D. Y. Kim, S. M. Jeong, J. W. Park, Macromol. Rapid Commun. 2008, 29, 729; b) S. Kidambi, C. Chan, I. Lee, Langmuir 2008, 24, 224.
19. J. Shi, L. Wang, Y. Chen, Langmuir 2009, 25, 6015.
20. V. Santhanam, R. P. Andres, Nano Lett. 2004, 4, 41.
21. a) A. Perl, D. N. Reinhoudt, J. Huskens, Adv. Mater. 2009, 21, 2257; b) B. Michel, A. Bernard, A. Bietsch, E. Delamarche, M. Geissler, D. Juncker, H. Kind, J. P. Renault, H. Rothuizen, H. Schmid, P. Schmidt-Winkel, R. Stutz, H. Wolf, IBM J. Res. Dev. 2001, 45, 697; c) H. Lalo, J. C. Cau, C. Thibault, N. Marsaud, C. Severac, C. Vieu, Microelectron. Eng. 2009, 86, 1428.
22. a) A. Benahmed, R. Lam, N. Rechner, C. M. Ho, J. Micro-Nanolithog. MEMS MOEMS 2007, 6; b) Z. Zheng, J. W. Jang, G. Zheng, C. A. Mirkin, Angew. Chem. Int. Ed. 2008, 47, 9951.
23. a) L. J. Guo, Adv. Mater. 2007, 19, 495; b) S. Y. Chou, P. R. Krauss, P. J. Renstrom, Science 1996, 272, 85.
24. S. Lenhert, C. A. Mirkin, H. Fuchs, Scanning 2010, 31, 1.
25. S. Lenhert, A. Sesma, M. Hirtz, L. Chi, H. Fuchs, H. P. Wiesmann, A. E. Osbourn, B. M. Moerschbacher, Langmuir 2007, 23, 10216.
26. Q. Shen, T. N. Chang, L. Yu, IEEE Trans. Indust. Elec. 2008, 55, 3266.
27. Y. Wang, L. R. Giam, M. Park, S. Lenhert, H. Fuchs, C. A. Mirkin, Small 2008, 4, 1666.
28. M. Strobel, S. Corn, C. S. Lyons, G. A. Korba, J. Polymer Sci. Part A—Polym. Chem. 1987, 25, 1295.
29. F. Hamadi, H. Latrache, M. Zekraoui, M. Ellouali, J. Bengourram, Mat. Sci. Eng. C—Biomim. 2009, 29, 1302.
30. a) A. Bietsch, B. Michel, J. Appl. Phys. 2000, 88, 4310; b) K. G. Sharp, G. S. Blackman, N. J. Glassmaker, A. Jagota, C. Y. Hui, Langmuir 2004, 20, 6430.
31. E. J. Peterson, B. L. Weeks, J. J. De Yoreo, P. V. Schwartz, J. Phys. Chem. B 2004, 108, 15206.
32. L. Tavares, J. Kjelstrup-Hansen, H. G. Rubahn, Small 2011, 7, 2460.
33. E. ten Grotenhuis, W. J. M. vanderKemp, J. G. Blok, J. C. van Miltenburg, J. P. van der Eerden, Colloid Surf. B—Biointerfaces 1996, 6, 209.
34. A. Mendez-Vilas, A. B. Jodar-Reyes, M. L. Gonzalez-Martin, Small 2009, 5, 1366.
35. D. Ausserre, M. P. Valignat, Opt. Express 2007, 15, 8329.

1. A method comprising:
printing one or more lipid inks on a substrate using a topographically structured stamp, and
removing the stamp from the substrate to form a patterned substrate,
wherein the stamp comprises one or more recesses containing the one or more lipid inks prior to printing the one or more lipid inks on the substrate using a topographically structured stamp,
wherein the one or more recesses have one or more recess patterns,
wherein the patterned substrate comprises one or more patterned arrays of lipid multilayer structures, and
wherein the patterned arrays are based on the one or more recess patterns.

2. The method of claim 1, wherein the one or more lipid multilayer structures comprise two or more lipid multilayer structures.

3. The method of claim 2, wherein a first lipid multilayer structure of the two or more lipid multilayer structures comprises a first lipid material and wherein a second lipid multilayer structure of the two or more lipid multilayer structures comprises a second lipid material that is different from the first lipid material.

4. The method of claim 1, wherein at least one of the one or more lipid multilayer structures comprises a phospholipid.

5. The method of claim 1, wherein at least one of the one or more lipid multilayer structures comprises a mixture of two or more lipids.

6. The method of claim 1, wherein the method comprises spreading the one or more lipid inks in the one or more recesses of the topographically structured stamp prior to printing the one or more lipid inks on the substrate using a topographically structured stamp.

7. The method of claim 6, wherein the one or more lipid inks are spread in the one or more recesses by exposing the one or more lipid inks in the one or more recesses to an increase in humidity.

8. The method of claim 1, wherein the method comprises applying the one or more lipid inks to the topographically structured stamp prior to printing the one or more lipid inks on the substrate using a topographically structured stamp.

9. The method of claim 1, wherein removing the stamp from the substrate to form the patterned substrate comprises moving the stamp relative to the substrate.

10. The method of claim 1, wherein removing the stamp from the substrate to form the patterned substrate comprises moving the substrate relative to the stamp.

11. The method of claim 1, wherein the one or more recesses comprise one or more grooves.

12. The method of claim 1, wherein each of the one or more lipid inks is a neat lipid ink.

13. The method of claim 1, wherein the lipid multilayer structures comprise one or more gratings.

14. The method of claim 1, wherein the lipid multilayer structures are microstructures.

15. The method of claim 1, wherein the lipid multilayer structures are nano structures.

16. A patterned substrate made by the following method:
printing one or more lipid inks on a substrate using a topographically structured stamp, and
removing the stamp from the substrate to form the patterned substrate,
wherein the stamp comprises one or more recesses containing the one or more lipid inks prior to printing one or more lipid inks on a substrate using the topographically structured stamp,
wherein the one or more recesses have one or more recess patterns,
wherein the patterned substrate comprises one or more patterned arrays of lipid multilayer structures, and
wherein the patterned arrays are based on the one or more recess patterns.

17.-27. (canceled)

28. A method comprising:
spreading one or more lipid inks on a substrate using an edge of topographically structured brush to thereby form a patterned substrate comprising a patterned array of one or more lipid multilayer structures on the substrate,
wherein the brush comprises one or more recesses in a surface of the brush including the edge,
wherein the one or more recesses extend to the edge, and
wherein the one or more recesses have a recess pattern that shape the one or more lipid inks to form the patterned array of one or more lipid multilayer structures.

29. The method of claim 28, wherein the one or more lipid multilayer structures comprise two or more lipid multilayer structures.

30. The method of claim 29, wherein a first lipid multilayer structure of the two or more lipid multilayer structures comprises a first lipid material and wherein a second lipid multilayer structure of the two or more lipid multilayer structures comprises a second lipid material that is different from the first lipid material.

31. The method of claim 28, wherein at least one of the one or more lipid multilayer structures comprises a phospholipid.

32. The method of claim 28, wherein at least one of the one or more lipid multilayer structures comprises a mixture of two or more lipids.

33. The method of claim 28, wherein the one or more recesses comprise one or more grooves.

34. The method of claim 28, wherein each of the one or more lipid inks is a neat lipid ink.

35. The method of claim 28, wherein the lipid multilayer structures comprise one or more gratings.

36. The method of claim 28, wherein the lipid multilayer structures comprise one or more gratings.

37. The method of claim 28, wherein the lipid multilayer structures are microstructures.

38. The method of claim 28, wherein the lipid multilayer structures are nano structures.

39. A printed substrate made according to the method of claim 28.

40.-46. (canceled)

47. The patterned substrate of claim 16, wherein the one or more lipid multilayer structures comprise two or more lipid multilayer structures.

48. The patterned substrate of claim 47, wherein a first lipid multilayer structure of the two or more lipid multilayer structures comprises a first lipid material and wherein a second lipid multilayer structure of the two or more lipid multilayer structures comprises a second lipid material that is different from the first lipid material.

49. The patterned substrate of claim 16, wherein at least one of the one or more lipid multilayer structures comprises a phospholipid.

50. The patterned substrate of claim 16, wherein at least one of the one or more lipid multilayer structures comprises a mixture of two or more lipids.

51. The patterned substrate of claim 16, wherein the method comprises spreading the one or more lipid inks in the one or more recesses of the topographically structured stamp prior to printing the one or more lipid inks on the substrate using a topographically structured stamp.

52. The patterned substrate of claim 51, wherein the one or more lipid inks are spread in the one or more recesses by exposing the one or more lipid inks in the one or more recesses to an increase in humidity.

53. The patterned substrate of claim 16, wherein the method comprises applying the one or more lipid inks to the topographically structured stamp prior to printing the one or more lipid inks on the substrate using a topographically structured stamp.

54. The patterned substrate of claim 16, wherein removing the stamp from the substrate to form the patterned substrate comprises moving the stamp relative to the substrate.

55. The patterned substrate of claim 16, wherein removing the stamp from the substrate to form the patterned substrate comprises moving the substrate relative to the stamp.

56. The patterned substrate of claim 16, wherein the one or more recesses comprise one or more grooves.

57. The patterned substrate of claim 16, wherein each of the one or more lipid inks is a neat lipid ink.

58. The patterned substrate of claim 16, wherein the lipid multilayer structures comprise one or more gratings.

59. The patterned substrate of claim 16, wherein the lipid multilayer structures are microstructures.

60. The patterned substrate of claim 16, wherein the lipid multilayer structures are nano structures.

61. The patterned substrate of claim 16, wherein the lipid multilayer structures of at least one of the one or more patterned arrays of lipid multilayer structure have uniform heights.

62. The patterned substrate of claim 61, wherein the lipid multilayer structures of at least one of the one or more patterned arrays of lipid multilayer structures comprise a grating of parallel lines that are unconnected to each other.

63. The patterned substrate of claim 16, wherein the lipid multilayer structures of at least one of the one or more patterned arrays of lipid multilayer structures comprise a grating of parallel lines that are unconnected to each other.
